(19) United States
(12) Patent Application Publication
(10) Pub. No.: US 2026/0104333 A1
(43) Pub. Date: Apr. 16, 2026

(54) GAS MEASURING DEVICE AND GAS MEASURING METHOD FOR MEASURING A HIGH TARGET GAS CONCENTRATION

(71) Applicant: Dräger Safety AG $ Co. KGaA, Lübeck (DE)

(72) Inventors: Jürgen Osswald, Lübeck (DE); Tom Pöthig, LüBeck (DE)

(21) Appl. No.: 19/351,826

(22) Filed: Oct. 7, 2025

(30) Foreign Application Priority Data
Oct. 14, 2024 (DE) 102024129723.9

(51) Int. Cl. G01N 1/40; G01N 33/00

(52) U.S. Cl. CPC G01N 1/4022; G01N 33/0039; G01N 33/005

(57) ABSTRACT

The present disclosure relates to a gas measuring device and to a gas measuring process for measuring the concentration of a combustible target gas. In an oxidation measurement mode, a detector and a compensator are heated. The heated detector oxidizes combustible target gas, and the oxidation further heats the detector. The heated compensator compensates for the influence of ambient conditions on the detector. An overall detection variable which depends on the detector temperature and the compensator temperature is measured, and the target gas concentration is determined depending on the overall detection variable. In a heat conduction measurement mode, the compensator is heated, and combustible target gas cools the compensator more than the ambient air does. A compensator detection variable which depends on the compensator temperature is measured, and the target gas concentration is determined depending on the measured compensator detection variable.

### CROSS-REFERENCE TO RELATED APPLICATIONS

This application claims the priority of German Patent Application No. 102024129723.9, filed on Oct. 14, 2024, and titled “GAS MEASURING DEVICE AND GAS MEASURING METHOD FOR MEASURING A HIGH TARGET GAS CONCENTRATION”, which is hereby incorporated by reference in its entirety for all nonlimiting purposes.

### TECHNICAL FIELD

The present disclosure relates to a gas measuring device and to a gas measuring method which are configured to measure the concentration of a combustible target gas relatively reliably even if the target gas is present at a high concentration.

### BACKGROUND

Gas measuring devices comprising a detector and a compensator have become known. An electrically conductive segment of the detector is heated and oxidizes combustible target gas in a gas sample—of course only if the gas sample contains a sufficient amount of combustible target gas. The oxidation of the target gas releases thermal energy, which thermal energy further heats the detector segment. The temperature of the detector segment therefore correlates with the target gas concentration that is sought and to be measured. An indicator for the temperature of the detector segment is measured.

The compensator makes it possible to at least partially compensate for the influence of ambient conditions on the detector, in particular the respective influence of the ambient temperature, the ambient pressure, and the ambient humidity. It is possible, but thanks to the compensator not necessary, to measure at least one or even every relevant ambient condition. Ideally, the compensator does not oxidize any target gas, but reacts to ambient conditions in the same way as the detector. Such gas measuring devices are also referred to as “heat-tone sensors” or “catalytically active sensors”. The present disclosure also uses this principle. Often both the detector and the compensator are configured as so-called pellistors.

The inventors have internally identified the following problem: The temperature of the detector segment only correlates reliably with the sought target gas concentration for as long as the detector segment is still able to oxidize combustible target gas. If the target gas concentration is high, a situation may arise in which there is combustible target gas inside the gas measuring device, but no longer sufficient oxygen to oxidize this combustible target gas. If the determination of the target gas concentration is even in this situation based on the overall detection variable and thus on the temperature of the detector segment, the gas measuring device may provide incorrect results. In particular, there is a risk that a high concentration of a combustible target gas will not be detected.

The present disclosure is based on the object of providing a gas measuring device and a gas measuring method which are capable of determining the concentration of a combustible target gas relatively reliably even if the target gas is present in a relatively high concentration.

### SUMMARY

The object is achieved by a gas measuring device having the features described herein and by a gas measuring method having the features described herein. Advantageous embodiments are specified in the claims. Advantageous embodiments of the gas measuring device according to the present disclosure are, where appropriate, also advantageous embodiments of the gas measuring method according to the present disclosure and vice versa.

The gas measuring device according to the present disclosure and the gas measuring method according to the present disclosure are configured to measure the concentration of a combustible target gas, wherein this target gas has appeared or can appear in a spatial region to be monitored. The target gas is, for example, methane (CH₄) or hydrogen (H₂). Generally, the gas measuring device according to the present disclosure and the gas measuring method according to the present disclosure provide an estimated value for the actual target gas concentration, wherein the estimated value may deviate from the actual (real) value.

It is possible for several combustible target gases to occur simultaneously in the spatial region. In this case, preferably the sum of the concentrations of these combustible target gases is measured. In the following, the term “target gas concentration” is used for short, even if several combustible target gases are present and the sum of the concentrations of these target gases is measured.

The gas measuring device can be operated in an oxidation measurement mode and also in a heat conduction measurement mode. More precisely: The gas measuring device can be operated in the oxidation measurement mode. The same gas measuring device can also be operated in the heat conduction measurement mode. These two modes are described in more detail below. Optionally, the gas measuring device can additionally be operated in an idle state.

The gas measuring device comprises a detector and a compensator. The detector comprises an electrically conductive detector segment. The compensator comprises an electrically conductive compensator segment. The gas measuring device is configured to apply a first voltage to the detector and a second voltage to the compensator. A voltage is applied to the compensator in both the oxidation measurement mode and the heat conduction measurement mode. A voltage is applied to the detector at least during operation in the oxidation measurement mode, and optionally but not necessarily also during operation in the heat conduction measurement mode. The voltage applied to the compensator can always be the same as the voltage applied to the detector. It is also possible for the two applied voltages to differ from each other, at least temporarily and/or in the heat conduction measurement mode. The voltage applied to the detector and/or the voltage applied to the compensator can be constant over time or vary over time. Preferably, the voltage is applied in a pulsed form in order to save on electrical energy. Preferably, in the optional idle state, no voltage is applied to either the detector or the compensator.

At least temporarily, a gas sample flows from the spatial region to be monitored into the interior of the gas measuring device, for example because the gas measuring device sucks in the gas sample and/or because the gas sample diffuses into the interior. At least a part of the gas sample reaches the detector, and at least part of the gas sample reaches the compensator.

For as long as a voltage is applied to the detector, electric current flows through the detector segment. This current heats the detector segment. The heating of the detector segment causes combustible target gas inside the gas measuring device to be oxidized. The oxidation releases thermal energy. This thermal energy increases the temperature of the detector segment through which the current flows. This effect occurs if the gas sample contains a sufficient amount of combustible target gas and if sufficient oxygen for oxidation is available. Otherwise, the situation may arise that, although combustible target gas is available, the detector does not oxidize it due to a lack of oxygen and for this reason no thermal energy is released.

As long as a voltage is applied to the compensator, electric current flows through the compensator segment. This electric current heats the compensator segment. The gas measuring device is configured such that, ideally, even while the compensator segment is heated, the compensator does not oxidize any combustible target gas inside the gas measuring device. This goal can only be approximately achieved for some combustible target gases, in particular for hydrogen.

The gas measuring device comprises an overall detection variable sensor. This overall detection variable sensor is configured to measure an overall detection variable. This overall detection variable depends not only on the temperature of the detector segment but also on the temperature of the compensator segment. In a first alternative, the higher the temperature of the detector segment is, the greater will be the overall detection variable, and the higher the temperature of the compensator segment is, the smaller will be the overall detection variable. Conversely, in a second alternative, the higher the temperature of the detector segment is, the smaller will be the overall detection variable and the higher the temperature of the compensator segment, the greater will be the overall detection variable. In one embodiment, the overall detection variable sensor is configured to measure a detector detection variable that depends on the temperature of the detector segment and to measure a compensator detection variable that depends on the temperature of the compensator segment, and the overall detection variable sensor is configured to derive the overall detection variable therefrom, for example as a difference.

Note: in the following, if reference is made to a sensor being configured to measure a physical variable, the following is meant: The sensor directly measures the physical variable or another variable which correlates with the physical variable to be measured, the other variable therefore being an indicator for the physical variable to be measured.

Ambient conditions, in particular the ambient temperature, the ambient humidity, and the ambient pressure, influence both the detector and the compensator. The ideal situation, in which the compensator reacts to the ambient conditions in the same way as the detector, can usually only be achieved approximately. For this reason, the overall detection variable is usually influenced not only by the target gas concentration, but also by ambient conditions. If sufficient oxygen is available, the overall detection variable will usually nevertheless be a good indicator for the target gas concentration.

For as long as there is still a sufficient amount of oxygen inside the gas measuring device, which the detector segment can use to oxidize combustible target gas, the overall detection variable will correlate with the sought target gas concentration. During operation in the oxidation measurement mode, the gas measuring device is configured to determine the target gas concentration depending on the measured overall detection variable. For example, in the oxidation measurement mode, the gas measuring device applies a given proportionality factor or another given functional dependency to at least one measured value of the overall detection variable. Optionally, the gas measuring device additionally uses a measured ambient condition, in particular the ambient temperature.

The gas measuring device comprises a compensator detection variable sensor. This compensator detection variable sensor is configured to measure a compensator detection variable. The compensator detection variable depends on the temperature of the compensator segment, and usually to a lesser extent on ambient conditions, but depends less and ideally not at all on the temperature of the detector segment. Preferably, the temperature of the detector segment influences the compensator detection variable at most half as much, particularly preferably at most a quarter as much, in particular at most a tenth as much as the temperature of the compensator segment influences it. In one example embodiment, the higher the temperature of the compensator segment, the greater will be the compensator detection variable and, in another example embodiment, the higher the temperature of the compensator segment, the smaller will be the compensator detection variable. It is possible that the compensator detection variable sensor is a part of the overall detection variable sensor. It is also possible that these two sensors are implemented by two different units which may be arranged spatially remote from each other.

In the heat conduction measurement mode, the gas measuring device determines the target gas concentration depending on the measured compensator detection variable and preferably does not use the overall detection variable for this determination. The gas measuring device applies a given functional relationship on the compensator detection variable. Often the measured target concentration is the larger, the smaller the compensation detection variable is. It is possible that the gas measuring device additionally uses the measured compensator detection variable to derive the overall detection variable in the oxidation measurement mode and/or to check the detector.

The operation in the heat conduction measurement mode exploits the fact that many combustible target gases, in particular methane and hydrogen, have a higher thermal conductivity than ambient air. The thermal conductivity is also called the thermal conductivity coefficient of a medium, describes the transport of heat without an active mass transport taking place, and has, for example, the measurement unit [W/m*K]. Generally, the respective thermal conductivity is known for each target gas under consideration. Due to the higher thermal conductivity, the heated compensator segment is cooled by a gas sample having a combustible target gas, compared to a state free of combustible target gas. For many target gases, therefore, the greater the target gas concentration in the gas sample, the lower will be the temperature of the compensator segment, provided the voltage applied to the compensator remains constant and with constant ambient conditions. For this reason, the temperature of the compensator segment and thus also the compensator detection variable correlate with the sought target gas concentration.

As already explained, the gas measuring device is configured to apply in both modes a voltage to the compensator segment. The gas measuring device is configured to apply the voltage as follows: The compensator segment is heated more by the applied voltage during operation in the heat conduction measurement mode than during operation in the oxidation measurement mode. Preferably, the temperature of the compensator segment during operation in the heat conduction measurement mode is at least 15%, particularly preferably at least 30%, in particular at least 50% higher than during operation in the oxidation measurement mode. This is valid for the same target gas concentration and the same ambient conditions.

In one embodiment, the temperature of the compensator segment during operation in the heat conduction measurement mode is at least 50° C., particularly preferably at least 100° C., and in particular at least 200° C., e.g. between 180° C. and 220° C., higher than during operation in the oxidation measurement mode. This stronger heating is preferably caused by the compensator segment absorbing more electrical power, for example because in the heat conduction measurement mode the applied voltage and/or the electric current intensity are greater than in the oxidation measurement mode or because the voltage is applied in pulsed form and the pulse duration is longer and/or the pause between two pulse durations is shorter. If the voltage is applied in pulsed form, the absorbed electrical power is preferably understood to be the absorbed electrical power averaged over time.

As already explained, the operation in the heat conduction measurement mode exploits the fact that many combustible target gases cool the heated compensator segment more than ambient air does. This cooling effect correlates with the sought target gas concentration. The inventors have found in internal tests that the cooling effect and thus this correlation is generally greater the more the compensator segment is heated. Otherwise, more heating requires more electrical energy. For these reasons, the compensator segment is heated more during operation in the heat conduction measurement mode than in the oxidation measurement mode.

During operation in the oxidation measurement mode, however, the compensator should react to ambient conditions in approximately the same way as the detector. Ideally, the compensator segment does not oxidize any combustible target gas. Generally, the more the detector segment and the compensator segment are heated, the more combustible target gas they will oxidize. Preferably, the gas measuring device is operated as follows: During operation in the oxidation measurement mode, the temperature of the compensator segment does not deviate too much from the temperature of the detector segment, so that the compensator reacts to ambient conditions in approximately the same way as the detector.

For the reasons just mentioned, it is reasonable to heat the compensator segment less during operation in the oxidation measurement mode than during operation in the heat conduction measurement mode. This feature often has the following effect: During operation in the oxidation measurement mode, ambient conditions are compensated for relatively well. During operation in the heat conduction measurement mode, the cooling effect depends relatively strongly on the target gas concentration and on the compensator segment temperature. In addition, electrical energy is saved compared to an example embodiment in which the compensator segment is heated strongly in both modes. Saving electrical energy is particularly important if the gas measuring device is not or cannot be connected to a stationary power supply network during use.

The situation may arise that oxygen is still present inside the gas measuring device, but only so little that only a relatively small proportion of combustible target gas in the interior can be oxidized. In this situation, in particular during operation in the heat conduction measurement mode, two opposing influences can act on the temperature of the compensator segment, both of which depend on the target gas concentration:
Combustible target gas cools the heated compensator segment more than ambient air or any other gas mixture that is free of combustible target gas.
Generally, it is not possible to completely prevent the heated compensator segment from oxidizing combustible target gas. The oxidation releases thermal energy, which further heats the compensator segment.

Ideally, the heated compensator segment does not oxidize any combustible target gas at all, preferably in both modes. One embodiment of how this objective can at least approximately be achieved is described below, optionally in combination with other embodiments. In other words: The embodiment described below increases the reliability of the temperature of the compensator segment being much more dependent on the cooling effect of the combustible target gas than on the heating effect of the oxidation.

According to this embodiment, a passivation coating is applied to a compensator functional component of the compensator. The compensator functional component comprises the electrically conductive compensator segment, which is heated by the electric current flowing through it, and preferably electrical insulation around the compensator segment. The compensator functional component has, for example, the shape of a sphere or an ellipsoid or a plate and accommodates the electrically conductive compensator segment in its interior.

The passivation coating surrounds the compensator functional component, i.e. forms the outer surface of the compensator functional component, and therefore comes into contact with a gas sample inside the gas measuring device. The passivation coating ideally covers the entire compensator functional component, i.e. there are no gaps in the passivation coating. The passivation coating separates the gas sample from the compensator functional component, ideally completely. As a result, the passivation coating prevents the compensator functional component from coming into physical or chemical contact with the gas sample and ideally completely prevents the heated compensator segment from oxidizing combustible target gas. On the other hand, there is thermal contact between the gas sample and the compensator functional component and thus also thermal contact between the gas sample and the heated compensator segment, so that a change in the thermal conductivity of the gas sample has a measurable effect on the compensator segment and the compensator detection variable sensor is able to measure the change in thermal conductivity.

The passivation coating comprises a chemical compound. Preferably, this chemical compound comprises iodine (1), in particular an iodide or an iodate. The passivation coating may additionally comprise other components, in particular due to an impurity that can generally not completely be avoided. The proportion (share) of the chemical compound with iodine, measured in percent by weight (wt. %), in the passivation coating is at least 50%, preferably at least 80%, particularly preferably at least 95%.

The inventors have discovered in internal investigations that a passivation coating with this chemical compound and with this proportion in percent by weight is particularly effective in preventing to a relevant extent the undesirable event of the heated compensator segment oxidizing combustible target gas. This desirable preventive effect persists during a relatively long use of the gas measuring device, even if this use takes several days, weeks, or even months. Other possible chemical compounds for the passivation coating, on the other hand, do not achieve this desired preventing effect as reliably over a longer period of time. This desired effect also occurs with hydrogen (H₂) as the combustible target gas.

Particularly preferably, the chemical compound, of which at least 50 wt. % of the passivation coating consists, is an iodide or an iodate of an alkali metal or alkaline earth metal. Preferably, the passivation coating consists of at least 80 wt. % of the iodide or iodate. The alkali metal or alkaline earth metal is preferably potassium (K). The chemical compound is particularly preferably potassium iodide (KI) or potassium iodate (KIO₃). These two chemical compounds have proven in internal tests to be particularly suitable for preventing relevant oxidation of combustible target gas over a long period of use. Internal tests have shown that this desired effect is also achieved for hydrogen as the target combustible gas. It is also possible for the chemical compound to be a mixture of potassium iodide (KI) and potassium iodate (KIO₃).

A preferred embodiment of the present disclosure is described below, which can be implemented in conjunction with the passivation coating just described or in conjunction with a compensator of a different design.

According to the present disclosure, both the detector segment and the compensator segment are heated while an electric current flows through these two segments. Preferably, the detector segment is heated to a high temperature. In order to reliably oxidize all combustible target gases that may occur, this temperature is preferably between 450° C. and 550° C. If only hydrogen (H₂) can appear as the combustible target gas to be detected and is to be reliably oxidized, it is in many cases sufficient to heat the detector segment to a temperature between 150° C. and 250° C. Preferably, the compensator segment is heated in such a way that, during operation in the oxidation measurement mode, the temperature of the heated compensator segment deviates from the temperature of the detector segment by at most 150° C., preferably by at most 100° C., and in one implementation is lower than the temperature of the detector segment.

The gas measuring device applies a voltage not only to the detector segment but also to the compensator segment. According to the present disclosure, the gas measuring device applies the voltage to the compensator segment in such a way that the compensator segment is heated more during operation in the heat conduction measurement mode than during operation in the oxidation measurement mode. In one example embodiment, the gas measuring device applies the voltage to the detector segment in such a way that the detector segment is heated equally in both modes.

In a preferred embodiment, however, the gas measuring device applies the voltage to the detector segment in such a way that the following effect is caused: The detector segment is heated more during operation in the oxidation measurement mode than during operation in the heat conduction measurement mode, preferably at least twice as much. Particularly preferably, during operation in the heat conduction measurement mode, no voltage is applied to the detector segment, and the detector segment is ideally not heated at all. The embodiment in which the detector segment is heated less or even not at all during operation in the heat conduction measurement mode has in particular the following advantages for the oxidation measurement mode: There is less risk that substances will be deposited on the surface of the detector due to the heating and that the detector will therefore no longer be able to reliably oxidize combustible target gas during operation in the oxidation measurement mode (the detector will less be “poisoned” or “coked”). In addition, lower heating consumes less electrical energy.

According to the present disclosure, the gas measuring device applies a voltage to the compensator and causes the following effect: The compensator segment is heated more during operation in the heat conduction measurement mode than during operation in the oxidation measurement mode. Various embodiments of how this effect is achieved are described below. Several of these embodiments can be combined with one another.

In one example embodiment, an electrical bypass line is arranged parallel to the compensator. A controllable switch can either release or block (interrupt) the bypass line. If the switch releases the bypass line, a parallel connection is implemented. Only a portion of the electric current flows through the compensator and heats the compensator segment. A further portion of the electric current flows through the parallel bypass line and does not contribute to heating the compensator segment. As is known, the current intensity of the current flowing through the compensator segment and the current intensity of the current flowing through the bypass line depend on the electrical resistance of the compensator segment and on the electrical resistance of the bypass line. If the switch blocks the bypass line, the bypass line will have a theoretically infinite electrical resistance, and ideally the entire electric current will flow through the compensator and heat the compensator segment. The gas measuring device can control the switch as follows: The controlled switch blocks the bypass line during operation in the heat conduction measurement mode and releases it during operation in the oxidation measurement mode. As a result, the compensator segment is heated more in the heat conduction measurement mode than in the oxidation measurement mode.

In a variation or generalization of this embodiment, a controllable electrical resistor component is arranged in the bypass line. By controlling this component, a signal-processing control unit of the gas measuring device can change the electrical resistance of the resistor component. The greater the electrical resistance of the component is, the more the compensator segment is heated. The resistor component is controlled as follows: During operation in the heat conduction measurement mode, its electrical resistance is greater than during operation in the oxidation measurement mode. The embodiment with the switch just described can be considered as a special case of a variable electrical resistance.

In a further development of the embodiment with the bypass line parallel to the compensator or in an alternative, a further bypass line is arranged parallel to the detector. A further controllable switch either releases or blocks (interrupts) the further bypass line. The gas measuring device can control the switch in such a way that the following occurs: During operation in the heat conduction measurement mode, the further bypass line is released; during operation in the oxidation measurement mode, it is blocked. As a result, the detector segment is heated less during operation in the heat conduction measurement mode than during operation in the oxidation measurement mode. Instead of the further switch, a further controllable electrical resistor component with variable electrical resistance can also be arranged in the further bypass line.

In one embodiment, the gas measuring device is configured as follows: The intensity (amperage) of the electric current flowing through the detector segment differs at least temporarily from the intensity of the electric current flowing through the compensator segment. For example, the compensator is arranged parallel to the detector, and/or the detector and the compensator are electrically supplied by two different circuits. In the case of a pulsed voltage “the intensity” is to be understood as the averaged intensity.

In one implementation of this embodiment, the compensator segment is caused to heat more during operation in the heat conduction measurement mode than during operation in the oxidation measurement mode as follows:
During operation in the oxidation measurement mode, the voltage is applied continuously or pulsed to the compensator segment as follows: At least while no combustible target gas is present inside the gas measuring device, the temperature of the compensator segment deviates from the temperature of the detector segment by at most a given upper temperature threshold. The upper threshold is preferably at most 150° C., particularly preferably at most 100° C.
During operation in the heat conduction measurement mode, the voltage is applied and/or pulsed to the compensator segment as follows: At least while no combustible target gas is present inside the gas measuring device, the temperature of the compensator segment is at least as high as a given lower temperature threshold, independently of the temperature of the detector segment and also independently of any cooling effect of a combustible target gas. Preferably, in the heat conduction measurement mode, the temperature of the compensator segment is higher than the temperature of the detector segment.

The gas measuring device preferably achieves this effect by adjusting the respectively applied voltage or an electrical resistor component accordingly by means of a suitable control.

According to the present disclosure, the gas measuring device can be operated either in the oxidation measurement mode or in the heat conduction measurement mode. Preferably, the gas measuring device is configured to operate as follows: The gas measuring device measures the target gas concentration in the oxidation measurement mode for as long as sufficient oxygen is present such that the heated detector segment is able to oxidize the, i.e. all, combustible target gas in the gas sample. Only if this condition is not fulfilled with sufficient certainty, the gas measuring device automatically switches to the heat conduction measurement mode. One reason is the following: As a rule, the measured concentration values obtained by the gas measuring device during operation in the oxidation measurement mode are more reliable and/or more accurate than the measured values in the heat conduction measurement mode—but only for as long as there is still sufficient oxygen in the gas sample, i.e. inside the gas measuring device. In other words, the gas measuring device is operated in the oxidation measurement mode for as long as possible and only in the heat conduction measurement mode if the oxidation measurement mode no longer leads to sufficiently reliable measured values. In many cases, this embodiment in particular ensures that a target gas with a relatively low target gas concentration is detected more reliably than in another possible procedure that determines the mode in which the gas measuring device is operated.

According to one implementation of this preferred embodiment, an oxidation criterion is given in a computer-evaluable form. This oxidation criterion is given in such a way that it is fulfilled for as long as there is still sufficient oxygen inside the gas measuring device so that the heated detector segment is able to oxidize a combustible target gas in the interior, and for this reason the overall detection variable is suitable for determining the target gas concentration with sufficient reliability.

In a preferred implementation, the oxidation criterion is fulfilled at least if the following condition is fulfilled: The target gas concentration determined by the gas measuring device during operation in the oxidation measurement mode is lower than a given first upper concentration threshold. This first upper concentration threshold is given in such a way that there is always sufficient oxygen present at least if the target gas concentration is lower than this upper concentration threshold. In this preferred implementation, the interior of the gas measuring device is preferably permanently in fluidic communication (connection) with the spatial region to be monitored, so that oxygen can continuously flow into the interior.

According to this embodiment, the gas measuring device thus automatically switches to the heat conduction measurement mode if the target gas concentration, which is determined depending on the overall detection variable, is above the first upper concentration threshold.

After being switched on, i.e. initially, the gas measuring device is preferably first operated in the oxidation measurement mode. It is also possible for the gas measuring device to be operated initially in the heat conduction measurement mode. The gas measuring device preferably remains in the oxidation measurement mode and determines the target gas concentration depending on the overall detection variable for as long as sufficient oxygen is still present. As a rule, the gas measuring device is in this case also able to reliably detect a combustible target gas with a relatively small target gas concentration. As soon as the gas measuring device automatically detects that the oxidation criterion is no longer fulfilled, it automatically switches to the heat conduction measurement mode and measures the target gas concentration depending on the compensator detection variable. Preferably, the gas measuring device automatically switches back to the oxidation measurement mode as soon as the gas measuring device has detected that the oxidation criterion is fulfilled again.

Preferably, the first upper concentration threshold of the oxidation criterion is determined empirically in advance, preferably according to the following specification: As long as the target gas concentration has not yet reached or exceeded the first upper concentration threshold, the detector segment will be able to oxidize all further target gas with sufficient certainty because oxygen is available in sufficient quantities. As soon as the target gas concentration reaches or exceeds the first upper concentration threshold, the undesirable situation may occur that no longer enough oxygen is available. In this situation, the overall detection variable is no longer a reliable indicator for the target gas concentration. Preferably, the gas measuring device switches back to the oxidation measurement mode if the target gas concentration determined using the compensator detection variable has fallen below this first upper concentration threshold or another upper concentration threshold.

In one embodiment, while operated in the oxidation measurement mode, the gas measuring device is configured to determine the target gas concentration not only depending on the overall detection variable, but also depending on the compensator detection variable, e.g. by exploiting the larger thermal conductivity of the target gas. As a rule, the two estimated values of the actual target gas concentration obtained in these different ways differ from each other.

A comparison enables a consistency check. More precisely: In many cases, a comparison of these two estimated values makes it possible to check whether the detector is still functioning properly or is severely poisoned. “Poisoning” of a detector refers to the process in which harmful substances are deposited on a surface of the heated detector segment and for this reason the detector segment is no longer sufficiently able to oxidize a combustible target gas, even if sufficient oxygen is present. As a rule, the detector segment is heated more than the compensator segment, and for this reason the detector is poisoned faster than the compensator. In one embodiment, the gas measuring device is configured to automatically check itself for poisoning. In some cases, a comparison of these two estimated values also allows the gas measuring device to automatically check whether or not there is still enough oxygen present.

An embodiment has been described above in which the gas measuring device automatically switches from the oxidation measurement mode to the heat conduction measurement mode if a given oxidation criterion is not or no longer fulfilled. In one implementation, the oxidation criterion is at least not fulfilled if the following condition has occurred: The target gas concentration, which is determined on the basis of the overall detection variable, is above a given first concentration threshold, specifically for at least one measured value and preferably for all measured values within a time period that is at least as long as a given minimum time period. Preferably, the minimum time period is given such that, even with a high concentration of combustible target gas, at least the minimum time period elapses until all combustible target gas inside the gas measuring device is oxidized, even if no oxygen flows in.

In an implementation of this embodiment, a second upper concentration threshold is given. The second upper concentration threshold is greater than the first upper concentration threshold. The implementation uses the embodiment just described, in which the gas measuring device, during operation in the oxidation measurement mode, additionally determines the target gas concentration on the basis of the compensator detection variable. The oxidation criterion is also no longer fulfilled, and the gas measuring device automatically switches to the heat conduction measurement mode, if the following condition is fulfilled: The target gas concentration, which is determined on the basis of the compensator detection variable, is above the given second upper concentration threshold. Preferably, the gas measuring device switches to the heat conduction measurement mode in any case if the condition dependent on the compensator detection variable is fulfilled, regardless of which target gas concentration the gas measuring device has determined depending on the overall detection variable.

This embodiment is a possible remedy in particular for the following situation: The target gas concentration in the spatial region to be monitored fluctuates (oscillates) greatly or can at least fluctuate greatly, for example because a user carries the gas measuring device into areas with different target gas loads or because a combustible target gas suddenly escapes or can escape (sharp increase) or a leak from which combustible target gas escaped is sealed (sharp decrease). The target gas concentration can also increase significantly if there is a large amount of combustible target gas in an enclosed container but not outside the container and a probe (sample) of the gas measuring device is inserted into the container from outside. The step in which the gas measuring device determines the target gas concentration based on the overall detection variable inevitably requires a certain amount of processing time. For this reason, in some situations, the gas measuring device may not be able to detect a suddenly occurring high target gas concentration quickly enough during operation in the oxidation measurement mode. The implementation with the second upper concentration threshold reduces the risk of this potentially dangerous event not being detected and therefore no alarm being issued. As a rule, the cooling effect of a large amount of combustible target gas can be quickly determined, even in the case of a sudden concentration change.

Preferably, the gas measuring device comprises a detector chamber. The detector is located in the detector chamber. The detector chamber is located inside the gas measuring device. At least a part of a gas sample from a spatial region to be monitored enters the detector chamber and thus the environment of the detector. Preferably, the compensator is located outside the detector chamber and/or is thermally separated from the detector in another or an additional way.

In one embodiment, the gas measuring device comprises an oxygen sensor. The oxygen sensor is configured to measure the content of oxygen in a gas sample located inside the gas measuring device. The oxygen sensor may be arranged to measure the oxygen content of a gas sample in the detector chamber. In many cases, the oxygen sensor can additionally measure the oxygen content of another gas sample that likewise originates from the spatial region to be monitored and likewise enters the interior of the gas measuring device, wherein this other gas sample has not yet entered the detector chamber and therefore no oxygen has yet been oxidized in this other gas sample. Such an oxygen sensor is often already available as a component of a gas measuring device.

The gas measuring device is configured to use at least one measured value of the oxygen sensor, preferably a measured time course of the oxygen content, to check whether or not the given oxidation criterion is fulfilled. Preferably, the oxidation criterion is fulfilled if the oxygen content in the detector chamber is above a given lower oxygen threshold for a sufficiently long period of time, and otherwise not. In one implementation, the lower threshold for the oxygen content is between 10 vol % and 15 vol % oxygen and particularly preferably 12 vol %. This lower oxygen threshold applies at least if the oxygen sensor measures the oxygen content in the other gas sample outside the detector chamber.

According to the present disclosure, the gas measuring device can be operated in the heat conduction measurement mode. In the heat conduction measurement mode, the gas measuring device determines the target gas concentration depending on the measured compensator detection variable. In one example embodiment, no voltage is applied to the detector segment permanently or at least temporarily while the gas measuring device is operated in the heat conduction measurement mode. This embodiment has the following advantages, in particular compared to an example embodiment in which a voltage is also applied to the detector segment in the heat conduction measurement mode:
As a rule, the gas sample located inside the gas measuring device and surrounding the detector has a high concentration of combustible target gas while the gas measuring device is being operated in the heat conduction measurement mode. If the detector segment were to oxidize combustible target gas even in this situation, the oxidation would release a particularly large amount of thermal energy, and the detector segment could be heated very strongly. This heating can lead to undesirable deposits on the detector segment (“coking”, “poisoning”) and put the detector out of operation.
The strongly heated detector segment could also heat the compensator segment, and this weakens the desired cooling effect of the combustible target gas to be detected.
In addition, because there is usually not enough oxygen inside the gas measuring device to oxidize all combustible target gas during operation in the heat conduction measurement mode, the strong heating can cause the detector to react chemically in an undesirable manner with the heated and only incompletely oxidized target gas. For example, the target gas could extract oxygen from a ceramic coating of the detector. This chemical reaction could damage the detector.
The process that the voltage applied to the detector segment heats the detector segment inevitably consumes electrical energy. As a rule, the gas measuring device is not, or at least not permanently, connected to a stationary power supply network, but comprises its own power supply unit. In particular in this case, it is important to consume relatively little electrical energy.

In one example embodiment, the gas measuring device comprises an actuating element which can be actuated by a user. The actuating element can be activated and deactivated. If the actuating element is activated, the gas measuring device switches to the heat conduction measurement mode and remains in this mode, regardless of whether the oxidation criterion is fulfilled or not, and also regardless of a measured target gas concentration and also regardless of an oxygen concentration. If the actuating element is deactivated, the gas measuring device switches to or remains in the oxidation measurement mode as described above or switches to or remains in the heat conduction measurement mode, preferably depending on whether the oxidation criterion is fulfilled or not. Preferably, the activation of the actuating element additionally ensures that no voltage is applied to the detector segment, i.e. that it is not heated.

In particular, in the following situations, it may be advantageous to use a user setting to cause the gas measuring device to be operated in the heat conduction measurement mode:
In the spatial region to be monitored, there is a gas that displaces oxygen, for example an inert gas, in particular nitrogen. This situation may have been deliberately created to prevent a fire or other oxidation in the spatial region. In this situation, there is often insufficient oxygen, and sometimes the gas measuring device is unable to detect this situation or is unable to detect it quickly. The situation may also have occurred unintentionally.
A high concentration of at least one combustible target gas is present or may be present in the spatial region to be monitored. During operation in the oxidation measurement mode, the detector would in this case be subjected to a heavy load. The high target gas concentration is usually detected relatively reliably even during operation in the heat conduction measurement mode.

In one application of the present disclosure, the gas measuring device is used to determine the concentration of hydrogen as the or a combustible target gas. Hydrogen is expected to become increasingly important, in particular as a low-emission energy source for drives or for generating electrical energy or as a starting material in the chemical industry. In one implementation, the gas measuring device is able to recognize (capture) a specification as to whether hydrogen or another combustible target gas should be detected. As already explained above, for the detection of hydrogen it is sufficient to heat the detector segment to a temperature below 200° C., while the detection of another combustible target gas usually requires a significantly higher temperature.

In one example embodiment, the gas measuring device according to the present disclosure has an output unit. The gas measuring device causes an alarm to be output on this output unit in at least one form perceivable by a human as soon as the gas measuring device has determined a target gas concentration outside a given value range, in particular above a given threshold value. Alternatively or additionally, the gas measuring device causes the determined target gas concentration itself to be output. In particular, the output unit is configured to output the alarm visually, acoustically and/or haptically (through vibrations). Optionally, the gas measuring device also causes the output unit to output the mode in which the gas measuring device is currently being operated. As a rule, the gas measuring device comprises its own power supply unit, and in one example embodiment the current state of charge of the power supply unit is additionally output on the output unit.

Such a gas measuring device can be carried by a person while this person is in a spatial region where combustible target gas may be present. It is also possible that the gas measuring device is located in this region while at least one person is carrying out work there, and is attached to a wall or ceiling or floor, for example.

In another example embodiment, the gas measuring device according to the present disclosure has a communication unit, but not necessarily its own output unit on which a measured target gas concentration is output. With the aid of this communication unit, the gas measuring device is able to transmit at least once a message to a spatially remote receiver, preferably repeatedly, wherein this message comprises information about a determined target gas concentration. The receiver is located, for example, in an operations center that remotely monitors a spatial region in which a combustible target gas may occur. Optionally, the message comprises information about the mode in which the gas measuring device is currently being operated. A display unit of the receiver is configured to outputting the received message in at least one form perceivable by a person. Such a gas measuring device can be installed so as to be stationary (fixed in place) in or near a spatial region to be monitored. Preferably, several such gas measuring devices are installed in or near this area.

A possible application of this other embodiment is the following: The gas measuring device—or at least one transducer of the gas measuring device—is arranged in a completely or at least largely enclosed space, for example in a boiler or in a pipe or in a container or in a room with a combustion system or in a storage room belonging to a building or to a vehicle. Initially, the enclosed space is blocked against access. The display unit of the receiver is arranged outside this enclosed space and outputs visually and/or acoustically a determined value for the target gas concentration. A user or a control unit releases access to the enclosed space if and while the determined target gas concentration is lower than a given concentration threshold and is therefore not dangerous for a person. The enclosed space is then “cleared”. For example, work in the enclosed space that could result in flying sparks may only be carried out if the target gas concentration is low enough.

In one implementation, the gas measuring device is arranged completely outside the enclosed space. A hose or other fluid guide unit connects the gas measuring device to the enclosed space. A pump or other fluid conveying unit of the gas measuring device draws (sucks in) a gas sample from the enclosed space through the fluid guide unit. Alternatively, the gas sample diffuses through the fluid guide unit to the gas measuring device.

In one embodiment, the gas measuring device is configured to control a spatially remote alarm unit. As soon as the measured target gas concentration is outside the permissible value range, the gas measuring device causes the alarm unit to output an alarm in at least one form perceptible to a human being, in particular acoustically.

### BRIEF DESCRIPTION OF THE DRAWINGS

The present disclosure will be described below on the basis of an exemplary embodiment. In the drawings,

FIG. 1 schematically shows a first embodiment of the gas measuring device, wherein the detector and the compensator are arranged in a Wheatstone bridge;

FIG. 2 shows two switches of the gas measuring device according to FIG. 1;

FIG. 3 shows an example of a detector configured as a pellistor;

FIG. 4 shows an example of a detector configured as a flat component;

FIG. 5 schematically shows a second embodiment of the gas measuring device; and

FIG. 6 shows examples of the detection variables used as a function of the target gas concentration.

### DETAILED DESCRIPTION

The gas measuring device according to the present disclosure and the gas measuring method according to the present disclosure are configured to monitor a spatial region for the presence of at least one combustible target gas and/or of at least approximately measuring the concentration of a combustible target gas in this area. The gas measuring device uses a principle known from the prior art to examine a gas sample from the spatial region for the presence and/or concentration of the combustible target gas.

A detector is located inside a housing of the gas measuring device. Through an opening in the housing, a gas mixture diffuses from the region to be monitored into the interior of the housing or is conveyed into the interior, e.g. sucked in by a pump. In the exemplary embodiment, the interior of the housing is permanently in fluidic communication with the region to be monitored during use, so that a gas sample continuously flows into the interior of the housing. As a rule, this gas sample contains the or each target gas to be detected, provided the target gas is present in the spatial region, and oxygen. It is also possible that the interior of the gas measuring device is only in fluidic communication with the spatial region during the process of conveying a gas sample into the interior.

The detector comprises an electrically conductive wire with a heating segment, the heating segment being referred to hereinafter as the detector segment. The detector segment, for example, is a coil that forms a segment of the wire. The electrically conductive material is, for example, platinum or rhodium or tungsten or an alloy using at least one of these metals. A voltage U is applied to this wire at least temporarily, so that an electric current I flows through the wire. The flowing current heats the detector segment, and the heated detector segment releases thermal energy. The released thermal energy causes at least one combustible target gas, as a rule any combustible target gas, inside the housing to be oxidized—of course only if the region and thus the interior contain at least one combustible target gas and if sufficient oxygen is available for oxidation.

In one application, methane (CH₄) is the or a combustible target gas to be detected. The addition of thermal energy causes methane to react with oxygen, producing water and carbon dioxide. CH₄ and 2 O₂ thus becomes 2 H₂O and CO₂. In another application, the target gas is hydrogen (H₂). As is well known, hydrogen reacts with oxygen to form water (H₂O).

While the target gas is oxidized, thermal energy is released inside the housing. This thermal energy acts on the detector and increases the temperature of the detector segment through which current flows. This temperature increase correlates with the released thermal energy and thus with the concentration of the target gas inside the housing. A gas measuring device with such a detector is sometimes referred to as a “heat-tone sensor”.

The increase in temperature changes a measurable property of the detector, wherein the property correlates with the temperature of the detector segment. For example, said temperature increase changes the electrical resistance R of the detector segment through which the current flows. For many electrically conductive materials, the electrical resistance R is known to be higher the higher the temperature of the conductive material. The gas measuring device measures at least one measurable variable which is influenced by the property and thus by the temperature of the detector segment and which is referred to below as the “detector detection variable”. The detector detection variable is, for example, directly the temperature or a variable that correlates with the electrical resistance R of the detector segment, for example the voltage U applied to the detector or the current I or the electrical power P absorbed by the detector segment.

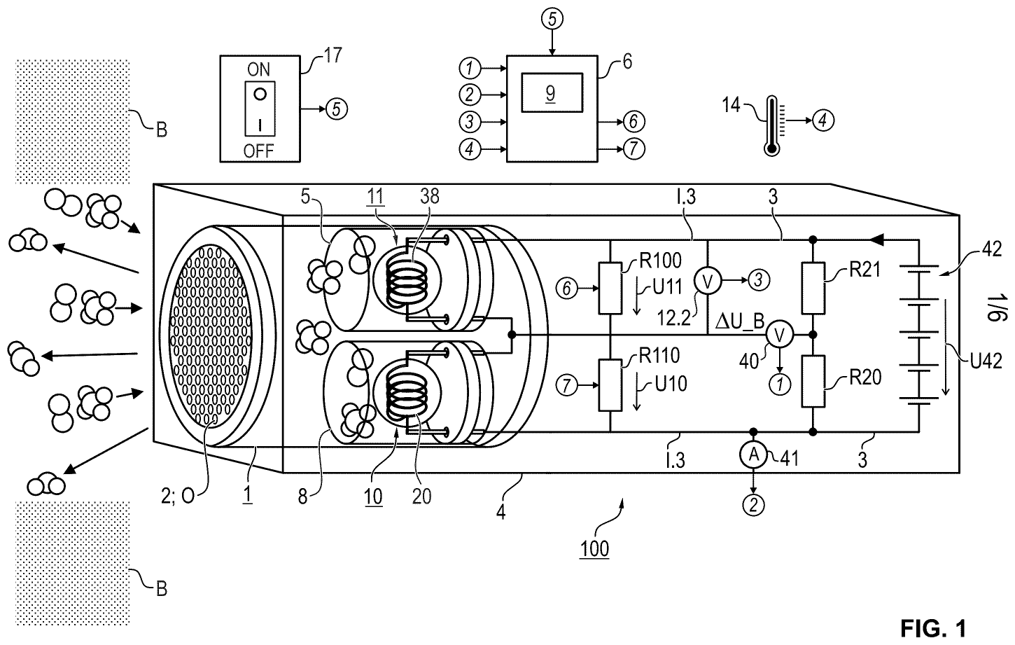

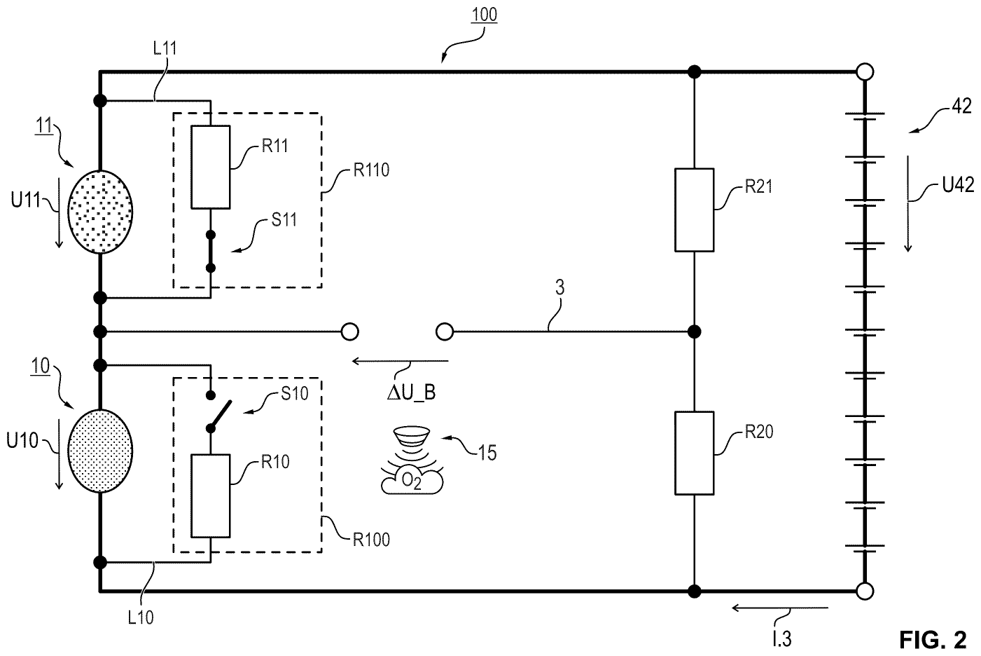

FIG. 1 and FIG. 2 show, by way of example, a first embodiment of a gas measuring device 100 according to the present disclosure. The same reference signs have the same meanings.

In the exemplary embodiment, a detector 10 is arranged in a detector chamber 8 and a compensator 11 is arranged in a compensator chamber 5; see FIG. 1. The detector chamber 8 with the detector 10 and the compensator chamber 5 with the compensator 11 are located in a sturdy housing 1. Thanks to an opening O, the sturdy housing 1 is in fluidic communication with the region to be monitored, so that a gas sample can pass from the region to be monitored into the interior of the housing 1 and from there also into the interior of the detector chamber 8. A flame protection means 2, for example a metallic grid, in the opening O reduces the risk of flames from the inside of the sturdy housing 1 spreading outwards. The sturdy housing 1 is surrounded by a schematically shown outer housing 4, which is preferably easy to grip and hold.

The voltage U10 applied to the detector 10 causes an electric current I to flow. The flowing current I heats a detector segment 20 of the detector 10 to a working temperature. If the heated detector segment 20 is to be capable of oxidizing all combustible target gases under consideration, this temperature will often lie between 450° C. and 550° C. In the case of hydrogen as the combustible target gas, a working temperature between 150° C. and 250° C. is often sufficient. However, this working temperature alone is generally not sufficient to oxidize a combustible target gas in the inner housing 1. A higher working temperature is often undesirable because it could lead to burning or even explosion of combustible target gas, which is often undesirable, and also consumes more electrical energy.

In order to be able to oxidize a combustible target gas despite a working temperature below 550° C. or, in the case of hydrogen even below 250° C., the detector 10 comprises a catalytic material which, in conjunction with the heated detector segment 20, oxidizes the target gas. For this reason, a gas measuring device with such a detector 10 is also referred to as a “catalytic sensor”.

In a frequently used implementation, the detector segment 20 is surrounded by electrical insulation, for example a ceramic casing. This electrical insulation electrically insulates the detector segment 20 and in particular prevents an undesired short circuit. The electrical insulation is thermally conductive so that the detector segment 20 can release thermal energy into the environment of the detector 10 and, conversely, thermal energy from the environment can further heat the detector segment 20. A coating of a catalytic material is applied to this electrical insulation. Alternatively, a catalytic material is embedded in the electrical insulation. This catalytic coating comes into contact with the gas mixture in the inner housing 1 and thus also with a combustible target gas. A detector 10 constructed in this way is often referred to as a “pellistor”.

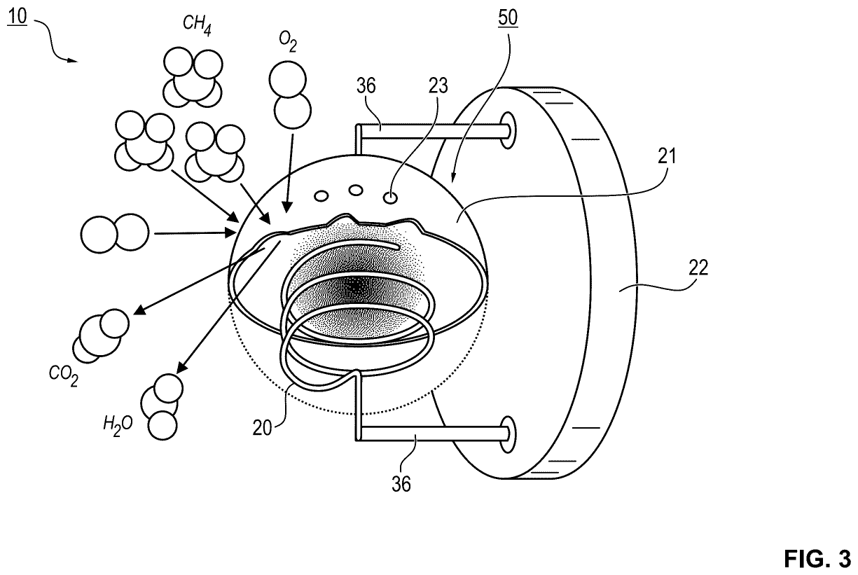

FIG. 3 shows an example of a detector 10 configured as a pellistor and schematically shows the conversion of methane (CH₄) into CO₂ and H₂O. The detector 10 comprises
a spirally wound and electrically conductive wire 20, which serves as the detector segment and is made of, for example, platinum or tungsten,
a ceramic casing 21, which surrounds the detector segment 20 and in the example shown has the shape of a solid sphere,
a catalytic coating on the outer surface of the ceramic casing 21, which coating is indicated in FIG. 3 by circles 23,
a mounting plate 22, and
electrical connections and mechanical supports 36 for the wire 20.

The detector segment 20, the ceramic casing 21, the mounting plate 22 and the connections and supports 36 belong to a functional component 50 of the detector 10. The catalytic coating 23 surrounds the detector functional component 50 or is embedded in the detector functional component 50.

Platinum or palladium, for example, is used as a catalytic material. Alternatively or in addition to the catalytic coating 23, catalytic material 23 can also be embedded in the ceramic casing 21.

In a preferred embodiment, the solid sphere of the detector 10 has a porous surface with a catalytic coating 23. In one example embodiment, this porous surface is produced as follows: the detector functional component 50, i.e. the detector 10 with the porous surface but without the catalytic coating, is provided. The catalytic coating 23 is applied to the porous surface, for example in an immersion bath, and a portion of the catalytic material penetrates into the interior of the detector 10. It is also possible for a ceramic material and a catalytically active substance to be mixed together and applied together to the detector segment 20, for example in an immersion bath.

Thanks to this porous surface, the detector 10 has a larger surface area compared to a smooth surface. Thanks to this larger surface area, the detector segment 20 is better able to oxidize combustible target gas, in particular because a larger amount of target gas comes into contact with the catalytic material. Thanks to the porous surface, a gas can reach deeper layers of the detector 10.

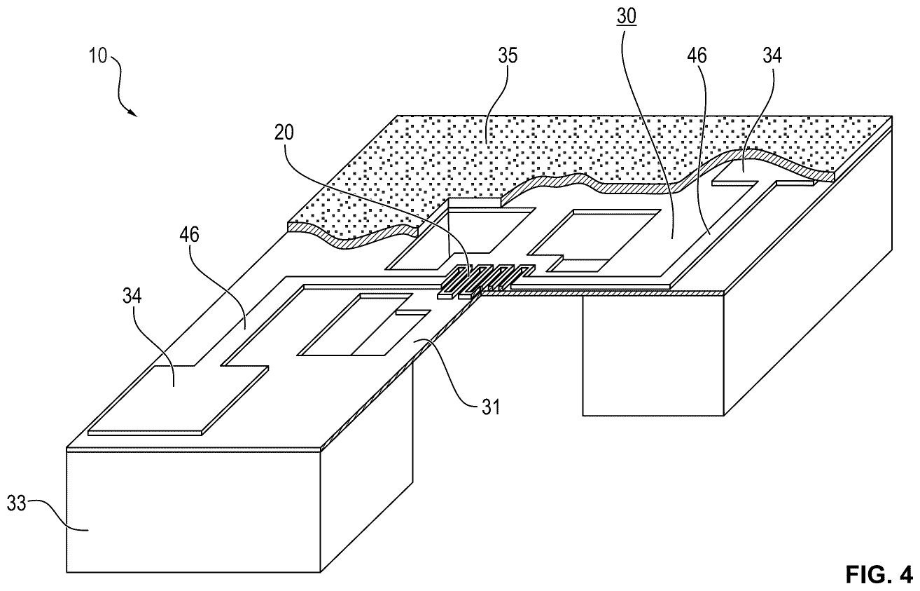

FIG. 4 shows a different example embodiment in which the detector 10 is configured as a flat component. The detector segment 20 is a component of an electrically conductive conductor track 30, which additionally has an electrical connection 46 and electrical contact points 34. The conductor track 30 is attached to a carrier plate 31. A wafer substrate 33 carries the carrier plate 31. A protective layer 35 is applied to the conductor track 30 with the detector segment 20. In one implementation, this protective layer 35 is catalytically effective. In another implementation, a catalytically active layer is applied to the protective layer 35, at least in the area of the detector segment 20. In a third possible implementation, the protective layer 35 is permeable to gas and covers a catalytically active layer on the detector segment 20.

In one example embodiment, the production of the detector 10 comprises the following steps:
The wire for the detector segment 20 of the detector 10 is provided.
To apply the ceramic coating to the wire, a liquid (coating suspension) is applied.
A heating current is applied to the detector segment 20. This causes the liquid to dry and calcine.

The liquid comprises the following three components:
a material that becomes catalytically active after drying, for example palladium nitrate [Pd(NO₃)₂] or hexachloroplatinic acid (H₂PtCl₆),
a carrier material, for example comprising an oxide of the elements aluminum, silicon, cerium and/or zirconium, and
a solvent, for example water.

The following description refers to both previously described implementations of the detector 10. However, the temperature of the detector 10 and thus also the detector detection variable are influenced not only by the thermal energy released but also by the ambient conditions in the region to be monitored, in particular the ambient temperature, as well as the humidity, the ambient pressure and the concentration of non-combustible gases, e.g. CO₂ or noble gases, in the air. These ambient conditions can also change the conditions inside the inner housing 1. These ambient conditions can also influence the detector temperature and thus the detector detection variable, for example because the thermal conductivity in the environment of the detector 10 is changed. It is desirable for the gas measuring device 100, despite varying ambient conditions, on the one hand to be able to reliably detect a combustible target gas and, on the other hand, to generate only relatively few false alarms, i.e. only relatively rarely decides that a target gas is present, although in reality no target gas has appeared above a detection threshold, which is an erroneous result.

In the exemplary embodiment, the gas measuring device 100 comprises an optional temperature sensor 14, which measures the ambient temperature in an environment of the gas measuring device 100; see FIG. 1. Preferably, the temperature sensor 14 measures the difference between the ambient temperature and a given reference temperature.

The gas measuring device 100 of the exemplary embodiment, however, comprises neither a sensor for the ambient pressure nor a sensor for the ambient humidity. The gas measuring device of the exemplary embodiment is also not necessarily capable of processing a signal from a sensor for the ambient pressure or from a sensor for the ambient humidity. Rather, the gas measuring device 100 constructively and/or computationally compensates to a certain extent for the influence of non-directly measured ambient conditions on that detection variable that depends on the temperature of the detector segment 20.

For this purpose, the gas measuring device 100 comprises a compensator 11 in addition to the detector 10; see FIG. 1. The compensator 11 also comprises a wire with a compensator segment. A voltage U11 is also applied to the compensator 11, so that electric current flows and the segment of the compensator 11 is also heated. The compensator 11 is also exposed to varying ambient conditions.

In a preferred implementation, the compensator 11 also comprises a spirally wound and electrically conductive wire, which serves as the compensator segment and is designated by the reference sign 38. The compensator 11 also comprises a ceramic casing, a mounting plate, electrical connections and mechanical supports. In contrast to the detector 10, however, the ceramic casing of the compensator 11 is not provided with a catalytic coating. The compensator segment 38, the ceramic casing, the mounting plate, the connections and the supports together belong to a compensator functional component 51, which can in particular have the shape of a sphere or a plate, i.e. can have the shape of the detector 10 of FIG. 3 or of the detector 10 in FIG. 4.

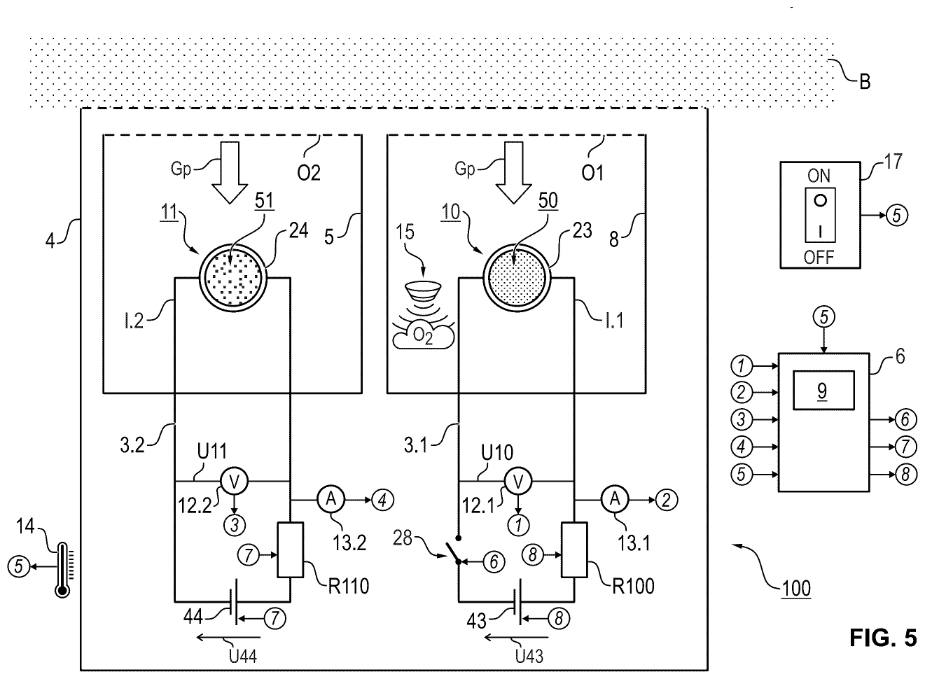

FIG. 1, FIG. 2 and FIG. 5 show the compensator 11 in the compensator chamber 5. It can be seen in FIG. 1 that the detector 10 comprises the detector segment 20 and the compensator 11 comprises a compensator segment 38. In the example in FIG. 1, FIG. 2 and FIG. 5, the compensator 11 is also configured as a spherical or ellipsoidal pellistor, but in contrast to the detector 10 according to FIG. 3, it does not comprise a catalytically active coating 23. The compensator 11 can also be configured as a flat component, like the detector 10 shown in FIG. 4.

In FIG. 1 and FIG. 2, the following further components of the gas measuring device 100 can be seen:
its own voltage source 42, for example a set of rechargeable batteries,
an electrical line 3 connecting the voltage source 42 to the detector 10 and to the compensator 11,
a voltage sensor 40 (FIG. 1 only),
a voltage sensor 12.2 (FIG. 1 only),
a current intensity sensor (ammeter) 41 (FIG. 1 only),
two electrical resistor components R100 and R110 with variable resistances,
two electrical resistor components R10 and R11 (FIG. 2 only),
two electrical resistor components R20 and R21,
an actuating element 17 (FIG. 1 only) and
a signal-processing control unit 6 (FIG. 1 only).

Optionally, a thermal barrier (not shown) inside the gas measuring device 100 thermally separates the detector 10 from the compensator 11. The present disclosure can also be implemented without such a thermal barrier.

The gas measuring device 100 according to FIG. 1 and FIG. 2 is constructed as a Wheatstone bridge. The voltage sensor 40 measures the bridge voltage ΔU_B in the Wheatstone bridge. This bridge voltage ΔU_B serves as the overall detection variable in the embodiment according to FIG. 1 and FIG. 2. In the first embodiment according to FIG. 1 and FIG. 2, the overall detection variable depends on the voltage U10 applied to the detector 10 and on the voltage U11 applied to the compensator 11 as follows: the higher the detector voltage U10, the greater will be the overall detection variable, and the higher the compensator voltage U11, the smaller will be the overall detection variable. The voltage sensor 12.2 measures the voltage U11 applied to the compensator 11. The current intensity sensor 41 measures the intensity I.3 of the current flowing through the line 3.

The compensator 10 and the detector 11 are connected in series in FIG. 1 and FIG. 2. The electrical resistor component R100 is connected in parallel to the detector 10; the electrical resistor component R110 is connected in parallel to the compensator 11. The electrical resistor components R20 and R21 are connected in parallel to the series connection formed by the detector 10 and the compensator 11.

FIG. 1 and FIG. 2 indicate
the voltage U42 of the voltage source 42,
the voltage U10 applied to the detector 10 and
the voltage U11 applied to the compensator 11.

The measured values from the sensors 40, 41, 12.2, 14 are transmitted to the control unit 6 and processed by the control unit 6. A signal-processing evaluation unit 9 derives an estimated value for the target gas concentration, in this case: for the concentration of methane or hydrogen, in the gas sample. In the exemplary embodiment, the evaluation unit 9 is a component of the control unit 6.

In the example shown in FIG. 1 and FIG. 2, the components form a Wheatstone bridge. The detector 10 and the compensator 11 are connected in series. The electrical resistance of the voltage sensor 40 is high compared to the electrical resistances of the components 10, 11, R10, R11, R20, R21. In one example embodiment, the voltage sensor 40 directly measures the so-called bridge voltage ΔU_B=(U10−U11)/2. By means of a closed-loop control, the current intensity I.3 is kept constant, so that the voltage U, in particular the bridge voltage ΔU_B, is proportional to the electrical resistance R and thus correlates with the temperature of the detector segment 20 and with the target gas concentration. For this closed-loop control, the actual current intensity I.3 measured by the current intensity sensor 41 is used.

In one implementation, a pulsed voltage is applied to save electrical energy. The control objective of keeping the current intensity I.3 constant refers to the current intensity during an electrical pulse. In another implementation, voltage is permanently applied to the detector 10 and to the compensator 11.

A corrected bridge voltage ΔU_Bₖₒᵣᵣ=ΔU_B−ΔU_B₀ correlates with the target gas concentration sought. Here, ΔU_B₀ is the zero point, i.e. the bridge voltage ΔU_B which occurs if no combustible target gas is present in the region to be monitored and thus inside the gas measuring device 100. This zero point ΔU_B₀ is preferably determined empirically in advance. The correction with the zero point ΔU_B₀ compensates for possible design-related differences between the detector 10 and the compensator 11. It is possible to determine the zero point ΔU_B₀ at least once again by means of at least a second adjustment during the service life of the gas measuring device 100.

In the embodiment according to FIG. 1 and FIG. 2, the corrected bridge voltage ΔU_Bₖₒᵣᵣ=ΔU_B−ΔU_B₀ serves as the overall detection variable.

FIG. 5 shows a second embodiment of the gas measuring device 100. Corresponding reference signs have the same meanings as in FIG. 1 and FIG. 2. The detector chamber 8 is in fluidic communication with the region B to be monitored via an opening O1, the compensator chamber 5 via an opening O2. The distance between the catalytic coating 23 and the rest of the detector 10 as well as the distance between a passivation coating 24 described below and the rest of the compensator 11 are shown exaggerated.

According to the second embodiment, the detector 10 and the compensator 11 are supplied with electrical energy independently of each other. A first electrical circuit I.1 connects the detector 10 to a first voltage source 43; a second electrical circuit 3.2 connects the compensator 11 to a second voltage source 44. An optional controllable switch 28, depending on its position, enables or interrupts the electrical circuit 3.1 between the detector 10 and the voltage source 43. The circuit 132 is preferably not interrupted.

A voltage sensor 12.1 measures the voltage U10 applied to the detector 10. A current intensity sensor 13.1 measures the intensity I.1 of the electric current flowing through the circuit 3.1 for the detector 10. A voltage sensor 12.2 measures the voltage U11 applied to the compensator 11. A current intensity sensor 13.2 measures the intensity I.2 of the electric current flowing through the circuit 3.2 for the compensator 11. The current intensities I.1 and I.2 are each kept constant by a closed-loop control.

In one implementation of the second embodiment, the overall detection variable is derived depending on the voltage difference ΔU=U10−U11. The voltage difference ΔU=U10−U11 is ideally equal to zero if no combustible target gas is present, but in practice it is also different from zero in the absence of combustible target gas. For this reason, a corrected voltage difference ΔUₖₒᵣᵣ=U10−U11−ΔU₀ is calculated and used as the overall detection variable. This overall detection variable ΔUₖₒᵣᵣ correlates with the target gas concentration. The zero value ΔU₀ occurs if no combustible target gas is present and again compensates for design-related differences between the detector 10 and the compensator 11. Again, it is possible to readjust the zero value ΔU₀ by making at least one new adjustment during the service life.

The procedure just described for measuring the target gas concentration requires the following: there must be sufficient oxygen in the detector chamber 8 so that the detector 10 is able to oxidize all combustible target gas. Only then can the thermal energy released during oxidation be reliably used as an indicator for the target gas concentration. If the target gas concentration is high, this condition may no longer be fulfilled. In particular, it is possible for combustible target gas to be present in the detector chamber 8, but no oxygen for oxidation. Oxygen can also be absent if another gas, which is not necessarily a target gas, has displaced the oxygen. This other gas is in particular an inert gas which is intended to prevent unwanted oxidation in the spatial region. However, if the target gas concentration is still only measured in this case using the thermal energy released during oxidation, there is a risk that the target gas concentration measured will be too low, i.e. a dangerously high target gas concentration will not be detected. This can endanger a user and is therefore undesirable.

For this reason, if the target gas concentration measured due to the released thermal energy has reached a given upper concentration threshold, a different procedure is preferably applied. The upper concentration threshold is selected such that if this upper concentration threshold is reached or exceeded, there is a possibility that almost all of the combustible target gas in the detector chamber 8 is oxidized. In other words: as long as the upper concentration threshold has not yet been reached, there will certainly still be sufficient oxygen in the detector chamber 8 for oxidation.

In the other procedure, i.e. if the target gas concentration has reached the upper concentration threshold, the detection variable that correlates with the temperature of the detector segment 20 is not used to determine the target gas concentration. In the exemplary embodiment, the voltage U10 applied to the detector 10 is therefore not used in the other procedure. The other procedure takes advantage of the fact that hydrogen and many other combustible target gases to be detected have a higher thermal conductivity than air. For this reason, these combustible target gases cool the heated compensator segment 38 of the compensator 11 more than ambient air does.

In the other procedure, the temperature of the compensator segment 38 is measured—more precisely: an indicator for the temperature. The temperature of the compensator segment 38 correlates with the target gas concentration sought. More precisely, compared to a state without a combustible target gas, the higher the target gas concentration, the lower the temperature, all other conditions remaining constant. In the exemplary embodiment, the current intensity I.3 (FIG. 1 and FIG. 2) or I.2 (FIG. 5) of the electric current flowing through the compensator 11 is kept constant by means of a corresponding closed-loop control. The voltage U11 applied to the compensator 11 correlates with the temperature of the compensator segment 38. Again, a zero point is determined empirically, namely the voltage U11₀, which is then applied to the compensator 11 if no combustible target gas is present. In the exemplary embodiment, the corrected compensator voltage U11ₖₒᵣᵣ=U11−U11₀ is used as the compensator detection variable.

The gas measuring device 100 can be operated in at least two different modes, optionally also in an idle state. The control unit 6 causes the following: the gas measuring device 100 of the exemplary embodiment automatically switches from one mode to the other mode, depending on a given oxidation criterion. In the exemplary embodiment, the gas measuring device 100 can be operated in the following modes:
in an oxidation measurement mode in which the gas measuring device 100 determines the target gas concentration depending on the corrected bridge voltage ΔU_Bₖₒᵣᵣ (embodiment according to FIG. 1 and FIG. 2) or depending on the corrected voltage difference ΔUₖₒᵣᵣ (embodiment according to FIG. 5), i.e. depending on the overall detection variable, or
in a heat conduction measurement mode in which the gas measuring device 100 determines the target gas concentration depending on the corrected compensator voltage U11ₖₒᵣᵣ=U11−U11₀, i.e. depending on the compensator detection variable.

In the exemplary embodiment, the corrected bridge voltage ΔU_Bₖₒᵣᵣ (embodiment according to FIG. 1 and FIG. 2) or the corrected voltage difference ΔUₖₒᵣᵣ (embodiment according to FIG. 5) therefore serves as the overall detection variable, and the corrected compensator voltage U11ₖₒᵣᵣ functions as the compensator detection variable.

As just explained, the gas measuring device 100 can be operated in two different modes. Preferably, different zero values are used in these two modes. The zero value ΔU_B₀ of the bridge voltage ΔU_B or the zero value ΔU₀ of the voltage difference ΔU=U10−U11 is used during operation in the oxidation measurement mode to calculate the overall detection variable. The zero value U11₀ of the compensator voltage U11 is used during operation in the heat conduction measurement mode to calculate the compensator detection variable U11ₖₒᵣᵣ. Note: the compensator 11 can have a different zero value U11₀ during operation in the oxidation measurement mode than during operation in the heat conduction measurement mode, in particular due to the different temperatures of the compensator segment 38.

During operation in the oxidation measurement mode, the evaluation unit 9 applies to the measured overall detection variable ΔU_Bₖₒᵣᵣ or ΔUₖₒᵣᵣ a first evaluation rule, which is given in a computer-evaluable form. This first evaluation rule depends on the overall detection variable ΔU_Bₖₒᵣᵣ or ΔUₖₒᵣᵣ and optionally on the measured ambient temperature and optionally on a further measured ambient condition. As explained above, an optional temperature sensor 14 is configured to measure the ambient temperature, preferably as a difference from a given reference temperature. During operation in the heat conduction measurement mode, the evaluation unit 9 applies to the measured compensator detection variable U11ₖₒᵣᵣ a second evaluation rule. The second evaluation rule depends on the compensator detection variable U11ₖₒᵣᵣ and optionally on the measured ambient temperature and/or a further measured ambient condition.

In the exemplary embodiment, with the aid of a sample the two evaluation rules are determined in advance by a learning procedure. For example, both evaluation rules are given, and each contains at least one model parameter. Preferably, a model parameter is the inverse value of an empirically determined proportionality factor, wherein this proportionality factor describes the influence of the target gas concentration on the respectively used detection variable. An optional further model parameter is the inverse value of a further empirically determined proportionality factor, wherein this further proportionality factor describes the influence of the measured ambient temperature on the detection variable used.

The oxidation criterion is given such that it is fulfilled at least if there is sufficient oxygen in the detector chamber 8 to oxidize all combustible target gas. Preferably, the oxidation criterion depends on the measured target gas concentration. Different implementations of the oxidation criterion are possible.

Preferably, the gas measuring device 100 is initially operated in the oxidation measurement mode. The control unit 6 repeatedly checks, preferably at a fixed sampling rate, whether the oxidation criterion is still fulfilled. For example, the evaluation unit 9 compares the target gas concentration determined in the oxidation measurement mode with a given first upper concentration threshold. Or the evaluation unit 9 checks how long the determined target gas concentration is above a given lower concentration threshold.

As soon as the control unit 6 has detected that the oxidation criterion is no longer fulfilled with sufficient certainty, the control unit 6 causes the gas measuring device 100 to automatically switch to the heat conduction measurement mode. In one implementation, the control unit 6 activates the switch S10 in FIG. 2 or the switch 28 in FIG. 5 and ensures that no more voltage is applied to the detector segment 20.

Preferably, during use the evaluation unit 9 continuously determines an estimated value for the target gas concentration on the basis of the compensator detection variable U11ₖₒᵣᵣ, even while the gas measuring device 100 is operated in the oxidation measurement mode. In one implementation, a second upper concentration threshold is given which is greater than the first upper concentration threshold. The control unit 6 causes the gas measuring device 100 to switch to the heat conduction measurement mode if the evaluation unit 9 has detected the following event: the target gas concentration, which is determined depending on the compensator detection variable U11ₖₒᵣᵣ, is above the second upper concentration threshold. The control unit 6 preferably causes this switching regardless of which target gas concentration has been measured depending on the overall detection variable.

As already explained, the oxidation criterion is fulfilled if sufficient oxygen is present in the detector chamber 8 for the detector 10 to be able to oxidize all combustible target gas present in said detector chamber. Embodiments have been described above in which the control unit 6 decides, depending on the measured target gas concentration, whether the oxidation criterion is fulfilled or not. It is also possible for an optional oxygen sensor 15 to measure the content of oxygen in the gas sample located in the detector chamber 8 and for the control unit 6 to check, depending on a measured value of the oxygen sensor 15, whether the oxidation criterion is fulfilled or not.

Preferably, the control unit 6 causes the gas measuring device 100 to switch back to the oxidation measurement mode if a given switch-back criterion is fulfilled.

In a preferred implementation, the switch-back criterion is fulfilled if the evaluation unit 9 has detected the following event: the target gas concentration, which has been measured in the heat conduction measurement mode, i.e. depending on the compensator detection variable U11ₖₒᵣᵣ, is lower than a given upper concentration threshold. This upper concentration threshold is preferably lower than the first upper concentration threshold mentioned above.

An embodiment has been described up to now in which the gas measuring device 100 automatically switches from one mode to the other mode. It is possible for the gas measuring device 100 to additionally comprise a selection switch, wherein a user can actuate this selection switch. By actuating the selection switch correspondingly, the user specifies how the gas measuring device 100 is to be operated:
The gas measuring device 100 is operated either in the oxidation measurement mode or in the heat conduction measurement mode, depending on the oxidation criterion.
The gas measuring device 100 is operated in the heat conduction measurement mode regardless of the oxidation criterion.

By way of example, a selection switch in the form of an actuating element 17 is shown in FIG. 1 and FIG. 5.

It is also conceivable that the control unit 6 has detected the following event, for example based on a detected user input: the detector chamber 8 has been flushed out with a gas sample that contains sufficient oxygen. For example, the user has carried the gas measuring device 100 into an area that has sufficient oxygen and is free of combustible target gas. For example, the user then actuates the actuating element 17, so that the oxidation measurement mode is possible again.

The following embodiment is also conceivable: the optional oxygen sensor 15 has measured a sufficiently high oxygen concentration in the detector chamber 8.

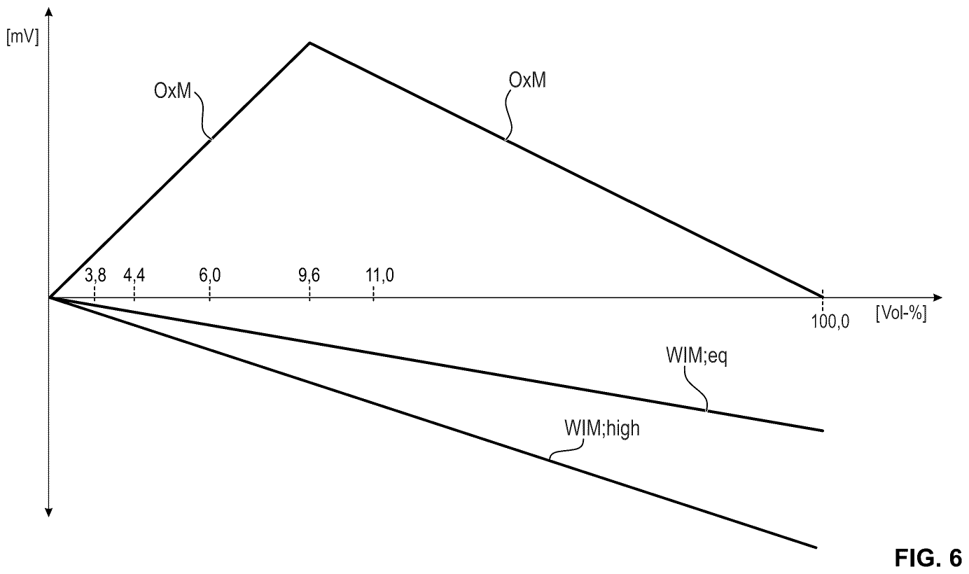

FIG. 6 shows schematically how the detection variable used in each case depends on the target gas concentration. Results of internal tests are shown. The concentration of the combustible target gas methane (CH₄) in [vol %] is plotted on the x-axis and the resulting value in [mV] for the detection variable used is plotted on the y-axis. The curve OxM refers to the oxidation measurement mode, in which the target gas concentration is determined depending on the overall detection variable, i.e. in this case depending on the corrected bridge voltage ΔU_Bₖₒᵣᵣ=ΔU_B−ΔU_B₀ (embodiment according to FIG. 1 and FIG. 2) or the corrected voltage difference ΔUₖₒᵣᵣ=U10−U11−ΔU₀ (embodiment according to FIG. 5). The curves WIM;eq and WIM;high refer to the heat conduction measurement mode, in which the target gas concentration is determined depending on the compensator detection variable, i.e. in this case depending on the corrected compensator voltage U11ₖₒᵣᵣ=U11−U11₀.

In the example in FIG. 6, methane is the target combustible gas. The lower explosion limit (LEL) in this example is 4.4 vol %. If the gas measuring device 100 has measured a target gas concentration greater than α*LEL, it will issue an alarm or cause a spatially remote receiver to issue an alarm. This applies regardless of the mode in which the target gas concentration was measured. The factor α lies between 0 and 0.6 and is preferably less than 0.5. In one implementation, the gas measuring device 100 issues a pre-alarm if the target gas concentration is greater than α₁*LEL, and a main alarm at a target gas concentration greater than α*LEL. Here 0<α₁<α<=0.6. For example, α₁=0.2 and α=0.4.

As already explained, in the exemplary embodiment, during use of the gas measuring device 100, the detector chamber 8 is permanently in fluidic communication with the spatial region B to be monitored, and a gas sample continuously flows into the detector chamber 8. The overall detection variable ΔU_Bₖₒᵣᵣ or ΔUₖₒᵣᵣ assumes the maximum value at a target gas concentration of 9.6 vol % of methane in air, which is the so-called stoichiometric concentration at which all oxygen in the detector chamber 8 is consumed. At a higher target gas concentration, the following two effects occur:
Because there is not enough oxygen in the detector chamber 8, the heated detector segment 20 oxidizes less combustible target gas, and the temperature of the detector segment 20 decreases again.
In addition, the gas sample in the detector chamber 8 and the gas sample in the compensator chamber 5 each have a greater thermal conductivity because methane has a higher thermal conductivity than air.

For these two reasons, the overall detection variable (the corrected bridge voltage ΔU_Bₖₒᵣᵣ; see FIG. 1 and FIG. 2, or the corrected voltage difference ΔUₖₒᵣᵣ; see FIG. 5) decrease again.

As already mentioned above, in a preferred embodiment, the control unit 6 causes the gas measuring device 100 to automatically switch to the heat conduction measurement mode if the target gas concentration determined in the oxidation measurement mode reaches or exceeds a given first upper concentration threshold. This first upper concentration threshold is lower than the stoichiometric concentration and is 6 vol % in the example shown.

Furthermore, an embodiment was mentioned above in which the control unit 6 causes the gas measuring device 100 operated in the heat conduction measurement mode to switch back to the oxidation measurement mode if a given switch-back criterion is fulfilled. This switch-back criterion is fulfilled if the target gas concentration determined in the heat conduction measurement mode is lower than a given switch-back threshold. In the example shown, this switch-back threshold is 3.8 vol %.

If the target gas concentration changes quickly during operation in the oxidation measurement mode, the aforementioned threshold of 6 vol % may not be sufficient in some cases. For safety reasons, a second upper concentration threshold of, for example, 11 vol % is given, i.e. larger than the stochiometric concentration. The control unit 6 causes the gas measuring device 100 to switch from the oxidation measurement mode to the heat conduction measurement mode if the following event is detected: the evaluation unit 9 determines, depending on the compensator detection variable U11ₖₒᵣᵣ, a target gas concentration above the second upper concentration threshold. The control unit 6 causes the switch to the heat conduction measurement mode regardless of which target gas concentration is determined in the oxidation measurement mode, i.e. depending on the overall detection variable ΔU_Bₖₒᵣᵣ or ΔUₖₒᵣᵣ.

During operation in the oxidation measurement mode, the compensator segment 38 must be heated to approximately the same temperature as the detector segment 20 so that the detector 10 and the compensator 11 react sufficiently similarly to ambient conditions, even to ambient conditions that are not directly measured, in order to at least approximately compensate for the influence of these ambient conditions on the overall detection variable ΔU_Bₖₒᵣᵣ or ΔUₖₒᵣᵣ. These ambient conditions comprise, in particular, the ambient pressure, the ambient humidity and the chemical composition of the ambient air. If the gas measuring device 100 does not have a temperature sensor 14, the ambient temperature will also be one of the non-measured ambient conditions.

In the exemplary embodiment, the compensator 11 should nevertheless not oxidize any combustible target gas, neither if the measured value for the target gas concentration is derived as a function of the detector voltage U10 and of the compensator voltage U11, nor if this measured value is derived only as a function of the compensator voltage U11. In particular, the compensator 11 must not oxidize any combustible target gas to a relevant extent if the target gas concentration is calculated as a function of the increased thermal conductivity and thus as a function of the compensator voltage U11. If the compensator 11 oxidizes even a relatively small amount of combustible target gas, this effect masks the effect of the increased thermal conductivity, i.e. the cooling, so that there is a high risk of an incorrect measured value being provided. The following undesirable effect is even possible: the oxidation of the target gas by the compensator 11 cancels out the increased thermal conductivity, so that the gas measuring device 100 provides the false result that no target gas is present.

In the exemplary embodiment, the wire for the compensator segment 38 of the compensator 11 is provided during the manufacture of the compensator 11. This wire is coated with a ceramic that is free of catalytically active material. In one implementation, aluminum oxide is used for this purpose.

As already explained, the wire of the compensator segment 38 is coated with ceramic. The ceramic is then coated with a passivation coating 24. The passivation coating 24 is applied as follows: the wire with the ceramic coating is immersed in an immersion bath containing a chemical composition and a solvent, e.g. water. The coated wire is then removed from the immersion bath and then dried. This causes the solvent to evaporate. While the gas measuring device 100 is used, the passivation coating 24 comes into contact with a gas sample in the compensator chamber 5. Ideally, the passivation coating 24 completely separates the ceramic and the wire of the compensator 11 from the gas sample.

In the exemplary embodiment, the passivation coating 24 consists of at least 50 wt. %, preferably at least 80 wt. %, particularly preferably at least 95 wt. %, of a chemical compound comprising iodine. Preferably, the passivation coating 24 consists of at least 50 wt. %, preferably at least 80 wt. %, of an iodide or an iodate of an alkali metal or an alkaline earth metal. This alkali metal or alkaline earth metal is preferably potassium. Particularly preferably, the chemical compound is potassium iodide (KI) or potassium iodate (KIO₃).

As already explained, the gas measuring device 100 can be operated either in an oxidation measurement mode or in a heat conduction measurement mode. In the oxidation measurement mode, the gas measuring device 100 determines the target gas concentration depending on the overall detection variable ΔU_Bₖₒᵣᵣ or ΔUₖₒᵣᵣ, in the heat conduction measurement mode depending on the compensator detection variable U11ₖₒᵣᵣ. In the oxidation measurement mode, the compensator 11 compensates for the influence of ambient conditions. For this reason, in the oxidation measurement mode, the compensator temperature does not deviate too greatly from the detector temperature, i.e. preferably by a maximum of 150° C., particularly preferably by a maximum of 100° C. This boundary determination does not exist in the heat conduction measurement mode because the compensator detection variable U11ₖₒᵣᵣ depends only on the compensator temperature.

The inventors have found in internal tests that in the heat conduction measurement mode, the higher the compensator temperature, the better the compensator detection variable U11ₖₒᵣᵣ will be an indicator for the target gas concentration sought. Background: as is well known, most target gases to be detected have a higher thermal conductivity than air, so the greater the target gas concentration, the more the compensator 11 will be cooled. The inventors have found internally that the higher the compensator temperature, the stronger the cooling effect. Compensator temperature refers to the temperature achieved by applying a voltage to the compensator 11 and an electric current flowing through the compensator segment 38.

By way of example, FIG. 6 illustrates the effect of two different temperatures of the compensator segment 38. The curve WIM;eq shows the dependence of the compensator detection variable, here U11ₖₒᵣᵣ, in the following possible implementation: not only in the oxidation measurement mode but also in the heat conduction measurement mode the compensator temperature is generated in such a way that it deviates only slightly from the detector temperature as described above and is the same in both modes. The curve WIM;high, in contrast, shows the dependence of the compensator detection variable U11ₖₒᵣᵣ if, according to the present disclosure, a higher compensator temperature is achieved in the heat conduction measurement mode than in the oxidation measurement mode. The curves shown in FIG. 6 are only to be understood as schematic.

The following technical teaching according to the present disclosure results from the principle just mentioned: the effect is that in the heat conduction measurement mode the compensator temperature is higher than in the oxidation measurement mode. After switching to the heat conduction measurement mode, the control unit 6 causes the intensity I.2, I.3 of the electric current flowing through the wire 38 of the compensator 11 to be increased. Conversely, the control unit 6 causes the current intensity I.2, I.3 to be reduced again after switching to the oxidation measurement mode.

Different implementations of how this is achieved are possible.

FIG. 2 shows a possible implementation that can be applied to the Wheatstone bridge shown in FIG. 1. A bypass line L11 with an electrical resistor component R11 and a switch S11 is arranged parallel to the compensator 11. The resistor component R11 and the switch S11 together form an implementation of the resistor component R110 in FIG. 1. If the switch S11 is closed, only a portion of the electric current I.3 will flow through the compensator 11 and heat the compensator segment 38. A further portion of the electric current I.3 flows parallel to the compensator 11 through the bypass line L11 with the closed switch S11. If the switch S11 is open, the entire electric current I.3 will flow through the compensator 11. One consequence is that if the switch S11 is open, the compensator segment 38 will be heated more than if the switch S11 is closed. The switch S11 can be controlled. The control unit 6 controls the switch S11 such that the switch S11 is open during operation in the heat conduction measurement mode and closed during operation in the oxidation measurement mode.

A further consequence is that if the switch S11 is closed, the electrical resistance of the circuit formed by the compensator 11 and the resistor component R11 will be lower than if the switch S11 is open. In one example embodiment, the voltage U11 applied to the compensator 11 is regulated such that the current intensity I.3 remains constant regardless of the position of the switch S11—of course after a settling time.

In another example embodiment, the voltage U11 is kept constant by a closed-loop control.

Optionally, a bypass line L10 with an electrical resistor component R10 and a switch S10 is connected in parallel to the detector 10. The resistor component R10 and the switch S10 together form an implementation of the resistor component R100 in FIG. 1. If the switch S10 is closed, only a portion of the current I.3 flows through the detector 10, and a further portion of the current I.3 flows through the bypass line L10. If the switch S10 is open, the entire electric current I.3 will flow through the detector 10. The control unit 6 controls the switch S10 in such a way that the following occurs: in the oxidation measurement mode, the switch S10 is open, and during operation in the heat conduction measurement mode, it is closed. This embodiment in particular reduces the risk of damage to the detector 10 while a high target gas concentration occurs in the detector chamber 8 and the gas measuring device 100 is therefore operated in the heat conduction measurement mode.

FIG. 5 shows an implementation which can be used if, as shown in FIG. 5, the detector 10 and the compensator 11 are supplied with electrical energy independently of one another and therefore the current intensity I.1 flowing through the detector 10 can deviate from the current intensity I.2 flowing through the compensator 11. The control unit 6 is configured to control the voltage source 43, preferably additionally the voltage source 44, independently of the voltage source 43. The control has the following effect: the voltage U11 applied to the compensator 11 is higher during operation in the heat conduction measurement mode than during operation in the oxidation measurement mode. Conversely, the voltage U10 applied to the detector 10 is preferably greater during operation in the oxidation measurement mode than during operation in the heat conduction measurement mode. In one example embodiment, the electrical resistance in the circuit 3.1 and that in the circuit 3.2 is the same in both modes.

According to the embodiment of FIG. 5, an electrical resistor component R110 with a variable resistance value is additionally arranged in the circuit 3.2 for the compensator 11. The control unit 6 is configured to control the resistor component R110. The control has the following effect: during operation in the oxidation measurement mode, the electrical resistance of the resistor component R110 is greater than during operation in the heat conduction measurement mode, preferably at least twice as high.

In a further development of this embodiment, an electrical resistor component R100 with a variable resistance value is arranged in the circuit 3.1 for the detector 10. The control unit 6 is configured to control the resistor component R100. The control has the following effect: during operation in the oxidation measurement mode, the electrical resistance of the resistor component R100 is lower than during operation in the heat conduction measurement mode, preferably at most half as high.

In another example embodiment, the control unit 6 is configured to control the voltage source 44 in the circuit 3.2 and optionally additionally the voltage source 43 in the circuit 3.1. By means of the control, the control unit 6 is able to change the voltage U44 of the voltage source 44 and optionally also the voltage U43 of the voltage source 43. By means of this control, the control unit 6 causes the different compensator temperatures in the two different modes.

It is possible that both the voltage U44 of the voltage source 44 and the electrical resistance of the resistor component R11 can be changed. It is also possible that only the voltage U44 or only the electrical resistance can be changed.

According to the embodiments described so far, the gas measuring device 100 is configured to automatically switch from the oxidation measurement mode to the heat conduction measurement mode and back again, preferably depending on whether the oxidation criterion is fulfilled or not. It is also possible for the gas measuring device 100 to be operated in the heat conduction measurement mode independently of the oxidation criterion. In one example embodiment, the gas measuring device 100 comprises an actuating element 17, which is shown schematically in FIG. 1 and in FIG. 5. The actuating element 17 can be activated and deactivated, for example by a user setting it to ON or OFF. The step of activating the actuating element 17 triggers the step of the gas measuring device 100 switching to the heat conduction measurement mode. Preferably, the detector 10 is then also deactivated, i.e. not supplied with electric current and therefore not heated. The step of deactivating the actuating element 17 triggers the step of the gas measuring device 100 switching to the oxidation measurement mode or remaining in the heat conduction measurement mode, depending on whether the oxidation criterion is fulfilled or not.

List of reference signs

| | |
|---|---|
| 1 | Inner housing, surrounds the detector chamber 8 and the |
| | compensator chamber 5 |
| 2 | Flame protection in the inner housing 1 |
| 3.1 | Circuit for the detector 10, comprises the voltage source 43 |
| | and the resistor component R10 |
| 3.2 | Circuit for the compensator 11, comprises the voltage |
| | source 44 and the resistor component R11 |
| 4 | Outer housing, surrounds the inner housing 1, the |
| | components R10, R11, R20, R21, S10, S11, the current |
| | intensity and voltage sensors and the voltage source 42, |
| | 43, 44 |
| 5 | Compensator chamber, surrounds the compensator 11, has |
| | the opening O2 |
| 8 | Detector chamber, surrounds the detector 10, has the |
| | opening O1 |
| 9 | Signal-processing evaluation unit, determines the target |
| | gas concentration, is in one example embodiment a |
| | component of the control unit 6 |
| 10 | Detector, comprises the detector functional component 50 |
| | with the detector segment 20 and the ceramic casing 21 as |
| | well as the catalytic coating 23 |
| 11 | Compensator, comprises the compensator functional |
| | component 51 with the compensator segment 38 and the |
| | ceramic casing 21 as well as the passivation coating 24 |
| 12.1 | Voltage sensor, measures the voltage U10 applied to the |
| | detector 10, belongs in the second example embodiment to |
| | the overall detection variable sensor |
| 12.2 | Voltage sensor, measures the voltage U11 applied to the |
| | compensator 11, operates as the compensator detection |
| | variable sensor and belongs in the second example |
| | embodiment to the overall detection variable sensor |
| 13.1 | Current intensity sensor (ammeter), measures the current |
| | intensity I.1 of the current flowing through the detector 10 |
| 13.2 | Current intensity sensor (ammeter), measures the current |
| | intensity I.2 of the current flowing through the compensator |
| | 11 |
| 14 | Optional temperature sensor, measures the difference |
| | between the ambient temperature and a given reference |
| | temperature |
| 15 | Optional oxygen sensor, measures the content of oxygen in |
| | the detector chamber 8 |
| 17 | Actuating element, with which a user can select operation |
| | in the heat conduction measurement mode |
| 20 | Electrically conductive detector segment, belongs to the |
| | detector functional component 50 |
| 21 | Ceramic casing around the detector segment 20 and |
| | around the compensator segment 38 |
| 22 | Mounting plate |
| 23 | Catalytically active coating on the ceramic casing 21 of the |
| | detector 10 |
| 24 | Passivation coating on the compensator functional |
| | component 51 of the compensator 11 |
| 28 | Switch that selectively allows or prevents electric current |
| | from flowing through the detector segment 20, sets the |
| | mode in which the gas measuring device 100 is operated |
| 30 | Electrically conductive conductor track, comprises the |
| | detector segment 20 |
| 31 | Carrier plate for the conductor track 30 |
| 33 | Wafer substrate |
| 34 | Electrical contact points for the conductor track 30 |
| 35 | Protective layer on the conductor track 30 |
| 36 | Mechanical supports for the detector segment 20 |
| 38 | Electrically conductive compensator segment, belongs to |
| | the compensator functional component 51 |
| 40 | Voltage sensor, measures the bridge voltage ΔU_B, |
| | operates in the first example embodiment as the overall |
| | detection variable sensor |
| 41 | Current intensity sensor, measures the current intensity I.3 |
| | in the Wheatstone bridge |
| 42 | Voltage source of the Wheatstone bridge, supplies the |
| | voltage U42 |
| 43 | Voltage source of circuit 3.1, supplies the voltage U43 |
| 44 | Voltage source of circuit 3.2, supplies the voltage U44 |
| 46 | electrical connection of the conductor track 30 |
| 50 | Functional component of the detector 10, comprises the |
| | heated detector segment 20 and the ceramic casing 21, |
| | surrounded by the catalytically active coating 23 |
| 51 | Functional component of the compensator 11, comprises |
| | the heated compensator segment 38 and the ceramic |
| | casing 21, surrounded by the passivation coating 24 |
| 100 | Gas measuring device, comprises the detector 10, the |
| | compensator 11, the temperature sensor 14, the control |
| | unit 6, the outer housing 4, the inner housing 1, the power |
| | supply unit 42, 43, 44 and the flame protection 2 |
| α | If the target gas concentration is greater than α*LEL, the |
| | gas measuring device 100 will issue a main alarm |
| α1 | If the target gas concentration is greater than α*LEL, the |
| | gas measuring device 100 will issue a pre-alarm |
| B | Spatial region to be monitored for combustible target gas |
| Gp | Gas sample from the spatial region B, enters the |
| | compensator chamber 5 and the detector chamber 8 |
| I.1 | Current intensity of the current flowing through circuit 3.1 |
| | and detector segment 20, measured by current intensity |
| | sensor 13.1 |
| I.2 | Current intensity of the current flowing through circuit 3.2 |
| | and compensator segment 38, measured by current |
| | intensity sensor 13.2 |
| I.3 | Current intensity in the Wheatstone bridge, measured by |
| | current intensity sensor 41 |
| L10 | Bypass line parallel to detector 10, contains the switch S10 |
| | and the resistor component R10 |
| L11 | Bypass line parallel to compensator 11, contains the switch |
| | S11 and the resistor component R11 |
| O1 | Opening in the detector chamber 8 |
| O2 | Opening in the compensator chamber 5 |
| O | Opening in the outer housing 4 |
| OxM | Detection variable as a function of the target gas |
| | concentration during operation in the oxidation |
| | measurement mode |
| R10 | Electrical resistor component, is connected in parallel or in |
| | series to the detector 10, belongs to the resistor component |
| | R100 |
| R11 | Electrical resistor component, is connected in parallel or in |
| | series to the compensator 11, belongs to the resistor |
| | component R110 |
| R20, R21 | Electrical resistor components parallel to the series |
| | connection formed by detector 10 and compensator 11 |
| R100 | Controllable electrical resistor component, is connected in |
| | parallel or in series to the detector 10, has a variable |
| | electrical resistance value, comprises in one implementa- |
| | tion the resistor component R10 and the switch S10 |
| R110 | Controllable electrical resistor component, is connected in |
| | parallel or in series to the compensator 11, has a variable |
| | electrical resistance value, comprises in one implementa- |
| | tion the resistor component R11 and the switch S11 |
| S10 | Controllable switch, which is connected in parallel to the |
| | detector 10, and belongs to the resistor component R100 |
| S11 | Controllable switch, which is connected in parallel to the |
| | compensator 11, and belongs to the resistor component |
| | R110 |
| U10 | Voltage applied to the detector 10, measured in one |
| | example embodiment by the voltage sensor 12.1 |
| U11 | Voltage applied to the compensator 11, measured in one |
| | example embodiment by the voltage sensor 12.2, serves as |
| | the compensator detection variable |
| U11₀ | Zero value of the compensator voltage U11, is used during |
| | operation in the heat conduction measurement mode |
| U11ₖₒᵣᵣ | Corrected compensator voltage, is equal to U11 − U11₀, |
| | serves as the compensator detection variable |
| U42 | Voltage of the voltage source 42 |
| U43 | Voltage of the voltage source 43 |
| U44 | Voltage of the voltage source 44 |
| ΔU | Non-corrected voltage difference, is equal to U10 − U11 |
| ΔU₀ | Zero value (zero point) of the voltage difference ΔU, is |
| | used during operation in the oxidation measurement mode |
| ΔUₖₒᵣᵣ | Corrected voltage difference, is equal to U10 − U11 − ΔU₀, |
| | serves as the overall detection variable |
| ΔU_B | Bridge voltage of the Wheatstone bridge, measured by the |
| | voltage sensor 40, is equal to (U10 − U11) / 2 |
| ΔU_B₀ | Zero value (zero point) of the bridge voltage ΔU_B, is |
| | used during operation in the oxidation measurement mode |
| ΔU_Bₖₒᵣᵣ | Corrected bridge voltage, is equal to ΔU_B − ΔU_B₀, |
| | serves as the overall detection variable |
| WIM; eq | Detection variable as a function of the target gas |
| | concentration during operation in the heat conduction |
| | measurement mode, wherein the compensator temperature |
| | is approximately equal to the detector temperature |
| WIM; high | Detection variable as a function of the target gas |
| | concentration during operation in the heat conduction |
| | measurement mode, wherein according to the present |
| | disclosure a higher compensator temperature is achieved |
| | in the heat conduction measurement mode than in the |
| | oxidation measurement mode |

1.-14. (canceled)

15. A gas measuring device for measuring a concentration of a combustible target gas, wherein the gas measuring device comprises:
a detector having an electrically conductive detector segment;
a compensator having an electrically conductive compensator segment;
an overall detection variable sensor; and
a compensator detection variable sensor,
wherein the gas measuring device is configured for a gas sample to at least temporarily flow from a spatial region to be monitored into an interior of the gas measuring device,
wherein the gas measuring device is configured to be operated in an oxidation measurement mode or in a heat conduction measurement mode,
wherein the gas measuring device is configured to:
at least while operating in the oxidation measurement mode, apply a first voltage to the detector segment such that the detector segment is heated; and
in both the oxidation measurement mode and the heat conduction measurement mode, apply a second voltage to the compensator segment such that the compensator segment is heated,
wherein the gas measuring device is further configured to apply the second voltage to the compensator segment in such a way that the compensator segment is heated more during operation in the heat conduction measurement mode than during operation in the oxidation measurement mode,
wherein the heating of the detector segment causes:
at least a part of the combustible target gas in a gas sample inside the gas measuring device to oxidize, and
the oxidation to increase a temperature of the detector segment,
wherein the overall detection variable sensor is configured to measure an overall detection variable that depends on the temperature of the detector segment and on a temperature of the compensator segment,
wherein the compensator detection variable sensor is configured to measure a compensator detection variable that depends on the temperature of the compensator segment, and
wherein the gas measuring device is configured to determine the concentration of the combustible target gas in the gas sample inside the gas measuring device:
based on the measured overall detection variable while the gas measuring device is operated in the oxidation measurement mode, and
based on the measured compensator detection variable while the gas measuring device is operated in the heat conduction measurement mode.

16. The gas measuring device of claim 15, wherein:
the compensator comprises a compensator functional component and a passivation coating,
wherein the compensator functional component comprises the electrically conductive compensator segment,
wherein the passivation coating:
surrounds the compensator functional component,
is located between the gas sample inside the gas measuring device and the compensator functional component, and
physically and chemically separates the gas sample from the compensator functional component, and
wherein the passivation coating comprises at least 50 percent by weight of a chemical compound comprising iodine.

17. The gas measuring device of claim 16, wherein the chemical compound comprises:
an iodide, or
an iodate of an alkali metal or of an alkaline earth metal.

18. The gas measuring device of claim 15, wherein the gas measuring device is configured:
to apply the first voltage to the detector segment in such a way that the detector segment is heated more during operation in the oxidation measurement mode than during operation in the heat conduction measurement mode.

19. The gas measuring device of claim 15, wherein:
the gas measuring device further comprises an electrical bypass line and a controllable switch for the bypass line,
wherein the bypass line is arranged parallel to the compensator, and
wherein the gas measuring device is configured to control the switch in such a way that the switch interrupts the bypass line during operation in the heat conduction measurement mode and releases the bypass line during operation in the oxidation measurement mode.

20. The gas measuring device of claim 15, wherein:
the gas measuring device is configured such that an intensity of an electric current flowing through the detector segment differs at least temporarily from an intensity of an electric current flowing through the compensator segment,
wherein the gas measuring device is configured to effect that:
during operation in the oxidation measurement mode, the temperature of the compensator segment does not deviate from the temperature of the detector segment by more than a given upper temperature threshold, and
during operation in the heat conduction measurement mode, the temperature of the compensator segment is at least as high as a given lower temperature threshold.

21. The gas measuring device of claim 15, wherein:
the gas measuring device is configured to automatically:
check whether a given oxidation criterion is fulfilled, wherein the oxidation criterion is fulfilled at least if there is still sufficient oxygen inside the gas measuring device for oxidation of the combustible target gas by the detector segment,
remain in the oxidation measurement mode as long as the oxidation criterion is fulfilled, and
switch to the heat conduction measurement mode if the oxidation criterion is no longer fulfilled.

22. The gas measuring device of claim 21, wherein the gas measuring device is configured such that the oxidation criterion is fulfilled at least if the target gas concentration, which is determined depending on the measured overall detection variable, is below a given first upper concentration threshold.

23. The gas measuring device of claim 22, wherein:
the gas measuring device is configured such that the oxidation criterion is additionally fulfilled at least if the target gas concentration, which is determined depending on the measured compensator detection variable, is below a given second upper concentration threshold,
wherein the second upper concentration threshold is greater than the first upper concentration threshold.

24. The gas measuring device of claim 21, wherein:
the gas measuring device further comprises a detector chamber and an oxygen sensor,
wherein the detector is arranged in the detector chamber,
wherein the oxygen sensor is configured to measure a content of oxygen in the gas sample while the gas sample is located inside the gas measuring device, and
wherein the gas measuring device is configured to check whether the given oxidation criterion is fulfilled depending on the measured oxygen content.

25. The gas measuring device of claim 15, wherein:
the gas measuring device further comprises an actuating element, and
wherein the gas measuring device is configured to, in response to activation of the actuating element:
switch to the heat conduction measurement mode, or
remain in the heat conduction measurement mode, and
wherein the gas measuring device can be operated either in the oxidation measurement mode or in the heat conduction measurement mode if the actuating element is deactivated.

26. The gas measuring device of claim 15, wherein the combustible target gas comprises hydrogen.

27. A gas measurement method for measuring a concentration of a combustible target gas, wherein the method is performed using a gas measuring device:
wherein the gas measuring device comprises:
a detector having an electrically conductive detector segment;
a compensator having an electrically conductive compensator segment;
an overall detection variable sensor; and
a compensator detection variable sensor,
wherein the gas measuring device is configured to be operated in an oxidation measurement mode or in a heat conduction measurement mode,
wherein the method comprises:
causing a gas sample to flow at least temporarily from a spatial region to be monitored into an interior of the gas measuring device; and
applying, in both the oxidation measurement mode and in the heat conduction measurement mode, a second voltage to the compensator segment such that the compensator segment is heated, wherein the second voltage is applied to the compensator segment in such a way that the compensator segment is heated more during operation in the heat conduction measurement mode than during operation in the oxidation measurement mode,
wherein, while the gas measuring device is operated in the oxidation measurement mode, the method further comprises:
applying a first voltage to the detector segment such that the detector segment is heated;
causing, based on the heating of the detector segment, at least a part of a combustible target gas in the gas sample inside the gas measuring device to oxidize,
wherein the oxidation increases a temperature of the detector segment;
measuring, using the overall detection variable sensor, an overall detection variable which depends on the temperature of the detector segment and on a temperature of the compensator segment; and
determining the concentration of the combustible target gas in the gas sample inside the gas measuring device based on the measured overall detection variable, and
wherein while the gas measuring device is operated in the heat conduction measurement mode, the method further comprises:
measuring, using the compensator detection variable sensor, a compensator detection variable which depends on the temperature of the compensator segment; and
determining the concentration of the combustible target gas in the gas sample inside the gas measuring device based on the measured compensator detection variable.

28. The gas measurement method of claim 27, wherein the second voltage is applied to the detector segment in such a way that the detector segment is heated more during operation in the oxidation measurement mode than during operation in the heat conduction measurement mode.

29. The gas measurement method of claim 27, wherein:
the compensator comprises a compensator functional component and a passivation coating,
wherein the compensator functional component comprises the electrically conductive compensator segment,
wherein the passivation coating:
surrounds the compensator functional component,
is located between the gas sample inside the gas measuring device and the compensator functional component, and
physically and chemically separates the gas sample from the compensator functional component, and
wherein the passivation coating comprises at least 50 percent by weight of a chemical compound comprising iodine.

30. The gas measurement method of claim 29, wherein the chemical compound comprises:
an iodide, or
an iodate of an alkali metal or of an alkaline earth metal.

31. The gas measurement method of claim 27, wherein:
the gas measuring device further comprises an electrical bypass line and a controllable switch for the bypass line,
wherein the bypass line is arranged parallel to the compensator, and
wherein the method further comprises controlling the switch in such a way that the switch interrupts the bypass line during operation in the heat conduction measurement mode and releases the bypass line during operation in the oxidation measurement mode.

32. The gas measurement method of claim 27, further comprising:
automatically, with the gas measuring device:
checking whether a given oxidation criterion is fulfilled, wherein the oxidation criterion is fulfilled at least if there is still sufficient oxygen inside the gas measuring device for oxidation of the combustible target gas by the detector segment;
remaining in the oxidation measurement mode as long as the oxidation criterion is fulfilled; and
switching to the heat conduction measurement mode if the oxidation criterion is no longer fulfilled.

33. The gas measurement method of claim 32, wherein:
the gas measuring device further comprises a detector chamber and an oxygen sensor,
wherein the detector is arranged in the detector chamber,
wherein the method further comprises:
measuring, with the oxygen sensor, a content of oxygen in the gas sample while the gas sample is located inside the gas measuring device; and
checking whether the given oxidation criterion is fulfilled depending on the measured oxygen content.

34. The gas measurement method of claim 27, wherein the combustible target gas comprises hydrogen.
